(19) United States
(12) Patent Application Publication
(10) Pub. No.: US 2008/0081333 A1
(43) Pub. Date: Apr. 3, 2008

(54) METHYLATED PROMOTERS AS BIOMARKERS OF COLON CANCER

(75) Inventors: Yuriko Mori, Baltimore, MD (US); Stephen J. Meltzer, Lutherville, MD (US)

Correspondence Address: SUGHRUE MION, PLLC, 2100 PENNSYLVANIA AVENUE, N.W., SUITE 800, WASHINGTON, DC, 20037, US

(73) Assignee: UNIVERSITY OF MARYLAND, BALTIMORE, Baltimore, MD (US)

(21) Appl. No.: 11/754,092

(22) Filed: May 25, 2007

Related U.S. Application Data
(60) Provisional application No. 60/808,833, filed on May 26, 2006.

(51) Int. Cl. C12Q 1/68

(52) U.S. Cl. 435/6

(57) ABSTRACT

The present invention provides methods for identifying or assessing probabilities for having or developing an abnormal condition in subject and for the recurrence of the abnormal condition in the subject after receiving treatment. The method comprises determining the methylation status of at least the tachykinin-1 (TAC1) gene in the subject and comparing this methylation status to normal methylation status. Differences between the methylation status of the TAC1 gene is indicative of the subject developing an abnormal condition or for the development or recurrence of the abnormal conditions after receiving treatment.

### CROSS-REFERENCE TO RELATED APPLICATIONS

This application claims priority to U.S. Provisional Application No. 60/808,833 filed 26 May 2006, which is incorporated by reference.

### STATEMENT REGARDING FEDERALLY SPONSORED RESEARCH OR DEVELOPMENT

Part of the work performed during development of this invention utilized U.S. Government funds under NIH Grants CA77057 and CA095323. The U.S. Government has certain rights in this invention.

### FIELD OF THE INVENTION

The present invention provides methods for identifying or assessing probabilities for having or developing an abnormal condition in subject and for the recurrence of the abnormal condition in the subject after receiving treatment. The method comprises determining the methylation status of at least the tachykinin-1 (TAC1) gene in the subject and comparing this methylation status to normal methylation status. Differences between the methylation status of the TAC1 gene is indicative of the subject developing an abnormal condition or for the development or recurrence of the abnormal conditions after receiving treatment.

### BACKGROUND OF THE INVENTION

Abnormal methylation of DNA (hypermethylation or hypomethylation) plays a role in gene activity, cell differentiation, tumorigenesis, X-chromosome inactivation, genomic imprinting and other major biological processes (See Razin, A., H., and Riggs, R. D. eds. in DNA Methylation Biochemistry and Biological Significance, Springer-Verlag, New York, 1984). In eukaryotic cells in general, methylation of cytosine residues that are immediately 5′ to a guanosine, occurs predominantly in cytosine-guanine (CG)-poor regions (See Bird, Nature, 321:209, 1986). In contrast, CG-rich regions (so-called “CpG islands”) are generally unmethylated in normal cells, except during X-chromosome inactivation and parental-specific imprinting (Li, et al., Nature, 366:362, 1993), where methylation of 5′ regulatory regions can lead to transcriptional repression. For example, a detailed analysis of the VHL gene showed aberrant methylation in a subset of sporadic renal cell carcinomas (Herman, et al., Proc. Natl. Acad. Sci., U.S.A., 91:9700, 1994).

The precise role of abnormal DNA methylation, however, in human tumorigenesis has not been fully established. About half of the tumor suppressor genes which have been shown to be mutated in the germline of patients with familial cancer syndromes have also been shown to be aberrantly methylated in some proportion of sporadic cancers, including APC, Rb, VHL, p16, hMLH1, and BRCA1 (reviewed in Baylin, et al., Adv. Cancer Res. 72:141-196 1998). Methylation of tumor suppressor genes in cancer is usually associated with (1) lack of gene transcription and (2) absence of coding region mutation. Thus CpG island methylation can serve as an alternative mechanism of gene inactivation (silencing) in human cancers.

Expression of a tumor suppressor gene can be diminished or ablated by de novo DNA methylation of a normally unmethylated CpG island (Issa, et al., Nature Genet., 7:536, 1994; Merlo, et al. Nature Med., 1:686, 1995 and Herman, et al., Cancer Res., 56:722, 1996). Methylation of tumor-suppressor genes leads to the reduced expression of tumor suppressor genes, resulting in unchecked cellular growth, tissue invasion, angiogenesis, and metastases (See Das, P. M. and Singal, R. J Clin Oncol, 22: 4632-4642 (2004) and Momparler, R. L. Oncogene, 22: 6479-6483 (2003)). Indeed, multiple studies have shown that promoter hypermethylation of tumor suppressor genes may also underlie carcinogenesis (See Eads, C. A., et al., Cancer Res., 61:3410-3418 (2001), Sato, F. et al. Cancer Res., 62: 6820-6822 (2002) and Takahashi, T., et al, Int J Cancer, 115:503-510 (2005), all of which are incorporated by reference). In addition, aberrant methylation across panels of genes correlates with prognosis in many cancers (See Darnton, S. J., et al., Int J Cancer, 115:351-358 (2005), Kawakami, K., et al., J Natl Cancer Inst, 92:1805-1811 (2000), Kikuchi, S., et al., Clin Cancer Res, 11:2954-2961 (2005) and Catto, J. W., et al., J Clin Oncol, 23:2903-2910 (2005), all of which are incorporated by reference). Indeed, prior studies have validated analyzing methylation patterns across a panel of genes to predict prognosis in esophageal and rectal cancers (See Brock, M. V., et al, Clin Cancer Res, 9:2912-2919 (2003), Ghadimi, B. M., et al, J Clin Oncol, 23:1826-1838 (2005), both incorporated by reference). Furthermore, human cancer cells typically contain nucleic acids that display somatic changes in DNA methylation (Makos, et al, Proc. Natl. Acad. Sci., USA, 89:1929, 1992; Ohtani-Fujita, et al., Oncogene, 8:1063, 1993).

Conversely, diminished DNA methylation (hypomethylation) has also been described in numerous human malignant and premalignant conditions (Martinez M E et al., Gastroenterology 2006 December; 131(6):1706-16; Cadieux B et al., Cancer Res. 2006 Sep. 1; 66(17):8469-76; Rodriquez J et al., Cancer Res. 2006 Sep. 1; 66(17):8462-8; Ehrlich M, Curr Top Microbiol Immunol 2006:310:251-74). This abnormally low level of methylation may lead to the activation, or abnormally high expression, of tumor-promoting genes or microRNAs, such as oncogenes and oncomiRs (Brueckner et al., Cancer Res. 2007 February 15:67(4):1419-23; Lujambio A et al., Cancer Res. 2007 February 15; 67(4):1424-9. Thus, there is a role for hypomethylation in the genesis and/or progression of human cancers.

Despite the abundance of evidence that characterizes certain molecular events in colorectal cancer initiation, promotion and progression, the incidence of colorectal cancer in the United States is rising. New tests and diagnostics are needed to better evaluate which patients are most at risk for developing colorectal polyps and cancers, or for the likelihood of their recurrence after initial treatment.

### SUMMARY OF THE INVENTION

The present invention provides methods for identifying or assessing probabilities for having or developing an abnormal condition in subject and for the recurrence of the abnormal condition in the subject after receiving treatment. The method comprises determining the methylation status of at least the tachykinin-1 (TAC1) gene in the subject and comparing this methylation status to normal methylation status. Differences between the methylation status of the TAC1 gene is indicative of the subject developing an abnormal condition or for the development or recurrence of the abnormal conditions after receiving treatment.

### BRIEF DESCRIPTION OF THE DRAWINGS

FIG. 1 depicts box plots of SST- and TACT-promoter methylation index (MI) for 17 normal colonic mucosae or 34 primary cancers. Methylation index (MI) represents the ratio of densely methylated DNA in the sample at the target sequence relative to the fully methylated positive control DNA. Primary colon cancers were significantly more methylated than were normal colonic mucosae for both SST and TALI (p<0.001 and p=0.01, Mann-Whitney's U-test).

FIG. 2 depicts line graphs displaying MI at the SST- and TACI-promoter regions for matching normal colonic mucosae and colon cancers from six patients. An increased promoter methylation level was observed for SST in all six (100%) and TAC1 in four (67%) of six cases.

FIG. 3 depicts plots that demonstrate the significant association between tumor microsatellite instability (MSI) status and SST promoter methylation (left panel) as well as Duke's stage and TAC1 promoter methylation (right panel). Each box plot represents the methylation index (MI) of each gene for each tumor group. SST methylation was significantly higher in 7 MSI-L cancers than in 27 non MSI-L cancers (12 MSI-H plus 15 MSS; p=0.02, Mann-Whitney's U-test). Similarly, TAC1 methylation was significantly higher in 13 Duke's A/B cancers than in 20 Duke's CID cancers (p=0.01, MannWhitney's U-test).

FIG. 4 depicts line graphs that demonstrate mRNA upregulation in association with decreased promoter methylation for SST and TAC1 induced by 5-aza-dC treatment in two colon cancer cell lines, HCT116 and HT29. Dashed and solid plots represent expression index (EI) and methylation index (MI), respectively. EI is mRNA expression level relative to a normal colonic mucosa specimen. Data normalization was performed using a CpG-free genomic sequence (MSP) and mRNA sequence (RT-PCR) of the beta-actin gene.

FIG. 5 depicts box plots of a methylation index (MI) at each gene promoter region for 17 normal colonic mucosae or 34 primary cancers. Primary colon cancers were significantly more methylated than normal colonic mucosae for NELL1 (p<0.001), CAVI (p<0.001), AKAP12 (p=0.01), ENG (p<0.01), and MAL (p<0.01). P values were calculated by Mann-Whitney's U-test.

FIG. 6 depicts plots of mRNA upregulation in association with decreased promoter methylation for each gene induced by 5-aza-dC treatment in two colon cancer cell lines, HCT116 and HT29. Dashed and solid plots represent expression index (EI) and methylation index (MI), respectively. A cell line was eliminated from analysis when the target gene promoter region was not methylated prior to 5-aza-dC treatment or failed to be demethylated by 5-aza-dC treatment.

FIG. 7 depicts a receiver-operator curve (ROC) analysis of normalized methylation value (NMV) of TAC1 for normal esophagus (NE) vs. esophageal adenocarcinoma (EAC) (A), NE vs. esophageal squamous cell carcinoma (ESCC) (B) and NE vs. malignant esophageal tissues (T) (C). The area under the ROC curve (AUROC) for the TAC1 gene conveys this gene's accuracy in distinguishing NE from EAC, ESCC and T in terms of sensitivity and specificity.

FIG. 8 (A) depicts that among 15 cases with corresponding NE, BE and EAC, one (No. 2) was unmethylated, three (Nos. 1, 3 and 13) were methylated only in EAC, three (Nos. 5, 16 and 17) were methylated only in BE, and the remaining nine were methylated in both BE and EAC. FIG. 8(B), depicts that in 41 cases with corresponding NE and T, four of four cases (No. 22, 23, 33 and 36) showing methylation in NE were also methylated in corresponding EAC.

FIG. 9 (A) depicts the mean normalized methylation value (NMV) of TAC1 was significantly higher in long-segment BE than in short-segment BE (p=0.00544, Student's t-test). (B) depicts TAC1 hypermethylation was associated with BE segment length (p=0.00256, Student's t-test).

FIG. 10 depicts the overall patient survival correlated with TAC1 methylation status in ESCC patients (A), but not in EAC patients (B). ESCC patients manifesting TAC1 hypermethylation had significantly shorter survivals than did patients without TAC1 methylation (mean, 22 months vs. 110 months, p=0.0102, Log-rank test).

FIG. 11 depicts 11 of 11 (3 EAC, 8 ESCC) esophageal cancer cell lines showing high normalized methylation values (NMV) of TAC1, which exceeded the cutoff value of 0.12. (B) KYSE 220 and BIC, which had the highest NMVs among the ESCC and EAC cell lines, respectively, were subjected to 5-Aza-dC treatment. After 5-Aza-dC treatment, the NMV of TAC1 was diminished, whereas the normalized mRNA value (NRV) of TAC1 was increased in both KYSE220 and BIC cell lines.

FIG. 12 depicts ROC curve analysis of SST NMVs for normal esophagus (NE) vs. esophageal adenocarcinoma (EAC) A, NE vs. esophageal squamous cell carcinoma (ESCC); B, NE vs. malignant esophageal tissues (T); C, The area under the ROC curve (AUROC) for the SST gene conveys this gene's accuracy in distinguishing NE from EAC, ESCC and T in terms of sensitivity and specificity.

FIG. 13 depicts that among 15 patients with matching NE, Barrett's esophagus (BE) and EAC, one (No. 2) was unmethylated in all tissues, one (No. 13) was methylated only in EAC, two (Nos. 14 and 16) were methylated only in BE, and the remaining eleven were methylated in both BE and EAC. (B) depicts that in 41 patients with corresponding NE and malignant esophageal tissue (T), five of five patients (Nos. 22, 24, 34, 36 and 39) showing methylation in NE were also methylated in corresponding malignant esophageal tissues (T). (C) depicts that SSTNMVs for T (mean=0.231) were significantly higher than those for matching NE (mean=0.055; p<0.0000001, Student's paired t-test).

FIG. 14 (A) depicts the mean normalized SST methylation value (NMV) was significantly higher in long-segment than in short-segment BE (p=0.001577, Student's t-test). (B) depicts that SST hypermethylation was positively associated with BE segment length (p=0.00073, Student's t-test).

FIG. 15 (A) depicts 12 of 12 (3 EAC, 9 ESCC) esophageal cancer cell lines showed high SST NMVs, exceeding the cutoff NMV level of 0.1. (B) depicts KYSE 220 and OE33, which had the highest NMVs among the ESCC and EAC cell lines, respectively, were subjected to 5-Aza-dC treatment. After 5-Aza-dC treatment in both KYSE220 and OE33 cell lines, the NMV of SST was diminished, whereas the normalized mRNA value (NRV) of SST was increased.

FIG. 16 depicts SST mRNA levels were significantly higher in EACs with unmethylated SST promoters than in EACs with methylated SST promoters (p=0.047, Student's t test).

FIG. 17 depicts a ROC curve analysis of AKAP12 NMVs of normal esophagus (N) vs. esophageal adenocarcinoma (EAC) (A) and ESCC vs. EAC (B). The area under the ROC curve (AUROC) conveys this biomarker's accuracy in distinguishing EAC from N and from ESCC in terms of its sensitivity and specificity.

FIG. 18 (A) depicts AKAP12 hypermethylation at apparent at relatively low levels, but nonetheless significantly higher in normal esophageal epithelium (N) from EAC patients (NEcA) (mean=0.00082) than in either normal esophagus from non-Barrett's/cancer patients (NE) (mean=0.00007; p=0.006) or normal esophagus from ESCC patients (NEcS) (mean=0.00021 and p=0.02). (B) depicts that AKAP12 NMVs in EAC (mean=0.1241) were significantly higher than those in matching N (mean=0.0008; p=0.0026, Student's paired t-test). (C) depicts AKAP12 NMVs in ESCC (mean=0.0018) did not differ significantly from those in matching NE (mean=0.0002) (p=0.27, Student's paired t-test).

FIG. 19 (A) depicts the mean normalized methylation value (NMV) of AKAP12 being significantly higher in long-segment BE (LSBE: 0.1879) than in short-segment BE (SSBE: 0.0543, p=0.047; Student's t-test). (B) depicts positive AKAP12 hypermethylation status being significantly correlated with BE segment length (p=0.045, Student's t-test).

FIG. 20 (A) depicts one of nine ESCC and two of three EAC esophageal cancer cell lines showing high AKAP12 methylation levels, above the threshold level of 0.05. (B) depicts BIC and OE33 EAC cells that were subjected to 5-Aza-dC treatment. After 5-Aza-dC treatment, the NMV of AKAP12 was diminished, while the normalized mRNA value (NRV) of AKAP12 was increased in both cell lines.

FIG. 21 depicts the AKAP12 mRNA levels in EACs with unmethylated AKAP12 promoters (mean=0.1663) being higher than those in EACs with methylated AKAP12 (mean=0.0668).

FIG. 22 depicts one-dimensional scatterplots that demonstrate normalized methylation ratio (NMR) for NELL1 (left panel) and MAL1 (right panel) in 41 normal and 47 cancerous gastric mucosae. Measurement was performed using rqMSP. Standardization specimen was fully methylated human genomic DNA. Internal control MSP amplicon was CpG-free β-actin. For both NELL1 and MAL, NMR in cancer was significantly higher than NMR in normal tissues. Asterisk indicates a sample whose NMR was off-scale in the graph (NMR=1.7). P-values were calculated by Student's t-test.

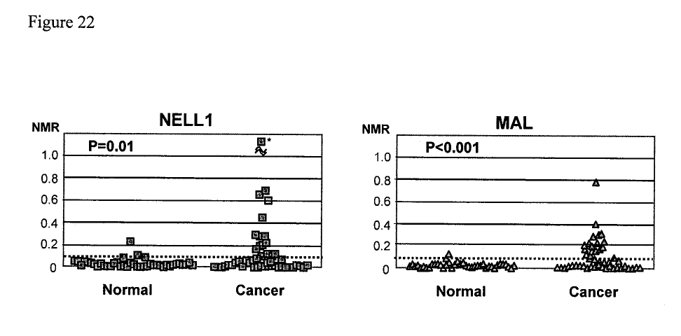

FIG. 23 depicts one-dimensional scatterplots that demonstrate normalized mRNA expression level for NELL1 (left panel) and MAL1 (right panel) in normal and cancerous gastric mucosae. Measurement was performed using real-time one-step quantitative PCR. Normalized expression ratio (NER) to a normal gastric mucosal specimen was measured for each gene using β-actin amplicon as the internal control. Five normal specimens were analyzed. Eleven and eight cancerous specimens were analyzed for NELL1 and MAL, respectively. Normal gastric mucosae demonstrated higher expression levels than cancers in both NELLL1 and MAL. P-values were calculated by Student's t-test.

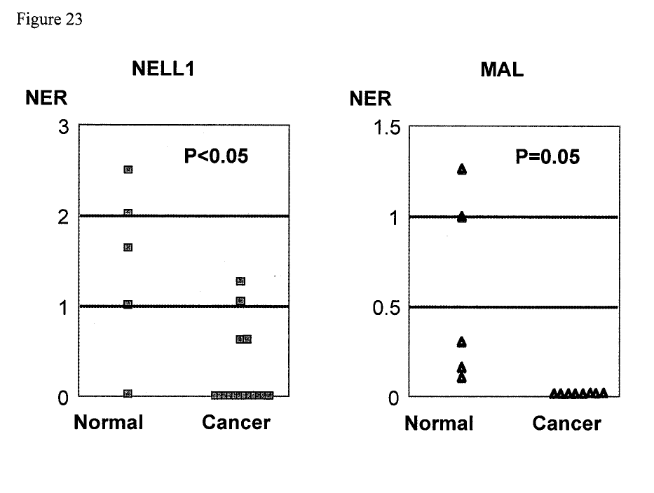

FIG. 24 depicts scatterplots that demonstrate normalized methylation ratio (NMR) for SST (left panel) and TAC1 (right panel) in 41 normal and 47 cancerous gastric mucosae and 4 gastric cancer cell lines. Measurement was performed using qMSP. For both SST and TAC1, NMR in cancers was significantly higher than NMR in normal tissues. P-values were calculated by Student's t-test

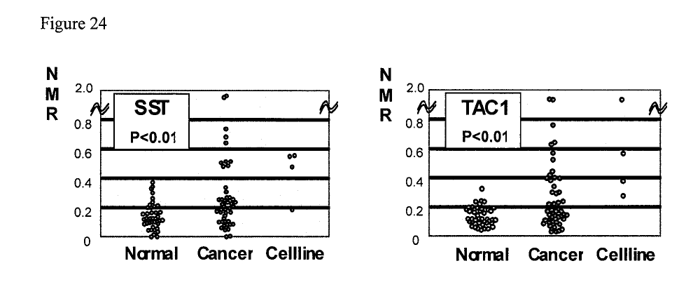

FIG. 25 depicts plots that demonstrate normalized expression ratio (NER) for SST (left) and TAC1 (right) mRNA in 6 normal (NM), 8 cancerous (PT) gastric tissues as well as 4 gastric cancer cell lines measured by qRTPCR. Both SST and TAC1 were downregulated in cancers relative to normal tissues.

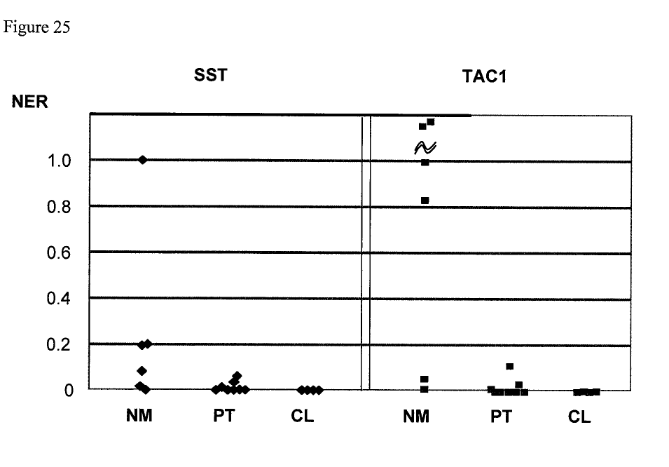

### DETAILED DESCRIPTION OF THE INVENTION

The present invention provides methods for identifying or assessing probabilities for the presence, recurrence or development of an abnormal condition in subject. As used herein, “predicting” or “assessing the probability” indicates that the methods described herein are designed to provide information to a health care provider or computer, to enable the health care provider or computer to determine the likelihood that an abnormal condition is already present, may occur in the future, or may recur in the future in a subject. Examples of health care providers include but are not limited to, an attending physician, oncologist, physician's assistant, pathologists, laboratory technician, etc. The information may also be provided to a computer, where the computer comprises a memory unit and machine-executable instructions that are configured to execute at least one algorithm designed to determine the likelihood that an abnormal condition may be already present, may occur in the future, or may recur in the future in a subject. Accordingly, the invention also provides devices for predicting the likelihood of current presence, future occurrence, or future recurrence of an abnormal condition in a subject, comprising a computer with machine-executable instructions for predicting the likelihood of presence, occurrence, or recurrence.

As used herein, the term “subject” is used interchangeably with the term “patient,” and is used to mean an animal, in particular a mammal, and even more particularly a non-human or human primate.

As used herein, a “recurrence” indicates that the abnormal condition occurs again in a patient, after the condition has been treated such that the condition is no longer detectable in the subject. The recurrence time for the abnormal condition resurfacing is not limited in any way. Furthermore, the term “treat” or “treatment” is used to indicate a procedure which is designed to ameliorate one or more causes, symptoms, or untoward effects of an abnormal condition in a subject. The treatment can, but need not, cure the subject, i.e., remove the cause(s), or remove entirely the symptom(s) and/or untoward effect(s) of the abnormal condition in the subject. The methods of the present invention can be performed prior to, in conjunction with, or after the treating the subject. Thus, for example, the methods of the present invention may be performed prior to treating the subject such that a more or less aggressive treatment strategy can be employed in the subject, if necessary. Accordingly, the present invention provides methods of individualizing treatments or therapeutic regimens in a subject by utilizing the methylation status or level of a gene or panel of genes. The phrase “therapeutic regimen” is used to indicate a procedure which is designed to terminate abnormal growth(s), inhibit growth and accelerate cell aging, induce apoptosis and cell death of neoplastic tissue within a subject. Additionally, “therapeutic regimen” means to reduce, stall, or inhibit the growth of or proliferation of tumor cells, including but not limited to precancerous or carcinoma cells. The therapeutic regimen may or may not be employed prior to performing the methods of the present invention. The invention is not limited by the therapeutic regimen contemplated. Examples of therapeutic regimens include but are not limited to chemotherapy (pharmaceuticals), radiation therapy, surgical intervention, endoscopic or colonoscopic excision, cell therapy, stem cell therapy, gene therapy and any combination thereof. In one embodiment, the therapeutic regimen comprises chemotherapy. In another embodiment, the therapeutic regimen comprises radiation therapy. In yet another embodiment, the therapeutic regimen comprises surgical intervention. In still another embodiment, the therapeutic regimen comprises a combination of chemotherapy and radiation therapy. In still another embodiment, the therapeutic regimen comprises initial or repeat colonoscopy with or without polypectomy or removal of other abnormal growths.

Of course, the therapeutic regimen that is being employed or contemplated will depend on the abnormal condition that the subject has or is suspected of having. As used herein, an “abnormal condition” is used to mean a disease, or aberrant cellular or metabolic condition. Examples of abnormal conditions in which the methods can be used include but are not limited to, dysplasia, neoplastic growth and abnormal cell proliferation. In one embodiment, the abnormal condition comprises neoplastic growth. In a more specific embodiment, the abnormal condition comprises a colon polyp. The colon polyp may or may not be cancerous. The invention, however, is not necessarily limited to the type of neoplasm. For example, the neoplasm may be a carcinoma of the digestive tract or any associated glands or organs, including, but not limited to, the throat, the salivary glands, vocal cords, esophagus, the stomach, the small intestine, the large intestine, the pancreas, liver, gallbladder, biliary tree, and rectum. Additional forms of neoplasms include, but are limited to, cancer of the lung, prostate, ovary, urinary tract, and breast.

The methods comprise determining the methylation status and level of a gene or panel of genes in the test subject, wherein the gene or panel of genes comprises at least the tachykinin-1 gene (TAC1). As used herein, “methylation status” is used to indicate the presence or absence or the level or extent of methyl group modification in the polynucleotide of at least one gene. As used herein, “methylation level” is used to indicate the quantitative measurement of methylated DNA for a given gene, defined as the percentage of total DNA copies of that gene that are determined to be methylated, based on quantitative methylation-specific PCR. As used herein, a “panel of genes” is a collection of genes comprising 2 or more distinct genes. In one embodiment, the panel of genes comprises at least 2, 3, 4, 5, 6, 7, 8, 9, 10, 11, 12, 13, 14, 15, 16, 17, 18, 19 or 20 or more genes.

The term “gene” is used similarly to as it is in the art. Namely, a gene is a region of DNA that is responsible for the production and regulation of a polypeptide chain. Genes include both coding and non-coding portions, including introns, exons, promoters, initiators, enhancers, terminators, microRNAs, and other regulatory elements. As used herein, “gene” is intended to mean at least a portion of a gene. Thus, for example, “gene” may be considered a promoter for the purposes of the present invention. Accordingly, in one embodiment of the present invention, at least one member of the panel of genes comprises a non-coding portion of the entire gene. In a particular embodiment, the non-coding portion of the gene is a promoter. In another embodiment, all members of the entire panel of genes comprise non-coding portions of the genes, such as but not limited to, introns. In another particular embodiment, the non-coding portions of the members of the genes are promoters. In another embodiment of the present invention, at least one member of the panel of genes comprises a coding portion of the gene. In another embodiment, all members of the entire panel of genes comprise coding portions of the genes. In one particular embodiment, the coding portion of the gene is at the 5′ end of the coding portion of the gene. In another particular embodiment, the coding portion of the gene is at the 3′ end of the coding portion of the gene.

Candidate members of the gene panel include, but are not limited to, tumor suppressor genes, tumor promoter genes and other genes that may be involved in cell cycle regulation. Examples of genes involved in the regulation of cell cycle that could serve as members of the gene panel include, but are not limited to, NELL1, APAKP12, SST, TAC1, SP, CA V-1 and ENG. Other genes involved in cell cycle regulation, such as, but not limited to p 6 will be recognized and appreciated by one of skill in the art.

In one embodiment, the gene or panel of genes comprises the tachykinin-1 gene (TAC1). TAC1 is a precursor for multiple hormones, including substance p (SP) and neurokinin A. These molecules affect secretion, motility, and inflammatory reactions of the gastrointestinal tract via activation of their receptors, neurokinin-1 and -2 receptors (NK1 and NK2). SP has been reported to be proliferative and antiapoptotic by activating mitogen-activated protein kinase cascade and NF-κB. SP has also been shown to mediate nonapoptotic programmed cell death. SP also potentiates cytotoxicity of lymphokine-activated-killer cells against colon cancer cells.

In another embodiment, the gene or panel of genes comprises TAC1 and somatostatin (SST). SST is a ubiquitously expressed hormone that regulates numerous endocrine systems, including the gastrointestinal tract. SST suppresses tumor growth indirectly through three known mechanisms: regulating release of mitogenic hormones and growth factors, inhibiting neoplastic angiogenesis, and modulating the immune system. SST directly suppresses cell growth in an autocrine manner, principally via SST receptor type 2, which is widely expressed in both normal and cancerous colonic epithelial cells.

Other candidate members of genes that may serve as members of the gene panel include, but are not limited to genes involved in angiogenesis. Examples of genes involved in angiogenesis include but are not limited to TIMP-1, TIMP-2, TIMP-3, TIMP-4, VEGF-A, VEGF-B, VEGF-C, VEGF-D, VEGF-E, IL-8, TGFβ and TGFα to name a few. One of skill in the art can recognize and appreciate genes involved in angiogenesis. In addition, the panel may also comprise angiogenic or growth facto receptors, such as, but not limited to, endoglin (ENG), which is a TGF receptor subunit gene.

Still other candidate member genes include, but are not limited to genes involved in DNA repair. Example of repair genes include, but are not limited to MGMT, BRCA1, BRCA2, hMLH1, hMSH1, hMLH6, and SHFM1 to name a few. One of skill in the art can recognize and appreciate DNA repair genes.

Additional candidate genes include, but are not limited to genes encoding receptors, growth factors and transcription factors to name a few. For example, T-cell maturation associated protein (MAL) is an endoplasmic reticulum membranous protein that participates in T-cell differentiation and T-cell receptor signaling. MAL appears to cooperate with caveolin-1 (CAV1) to coordinate diverse extracellular and intracellular signaling process. In particular, downregulation of CAV1 transforms cells via the p42/44 MAP kinase cascade activation.

Additional examples of a candidate for gene to serve on the panel include, but are not limited to, Hpp-1, sVEGFR-2 (sFLK-1), ESR1, IGFIR, IGFR, c-KIT, PDGFRα, HGFR, Grb2, bFGFR-2, FGFR-2, FGFR-3, AKAP12, PDEGF, RARBeta, and RASSF1A. Additional candidates include peptides containing epidermal growth factor like motifs, such as, but not limited to, NELL1 and NELL2. NELL1 is a protein kinase C-binding protein that is required for osteoblast differentiation. A kinase [PRKA] anchor protein [gravin] 12 (AKAP12) is a protein kinase A-C-binding protein that coordinates intracellular signaling of PKA and PKC.

In one embodiment, the panel of gene comprises a combination of at least 2, 3, 4 or 5 of the genes selected from the group consisting of TAC1, SST, NELL1, AKAP12, CAV1, ENG and MAL.

The invention is not limited by the types of assays used to assess methylation status of the members of the gene or gene panel. Indeed, any assay that can be employed to determine the methylation status of the gene or gene panel should suffice for the purposes of the present invention. In general, assays are designed to assess the methylation status of individual genes, or portions thereof. Examples of types of assays used to assess the methylation pattern include, but are not limited to, Southern blotting, single nucleotide primer extension, methylation-specific polymerase chain reaction (MSPCR), restriction landmark genomic scanning for methylation (RLGS-M) and CpG island microarray, single nucleotide primer extension (SNuPE), and combined bisulfite restriction analysis (COBRA). The COBRA technique is disclosed in Xiong, Z. and Laird, P., Nucleic Acids Research, 25(12): 2532-2534 (1997), which is incorporated by reference. In addition, methylation arrays may also be employed to determine the methylation status of a gene or panel of genes. Methylation arrays are disclosed in Beier V, et al., Adv Biochem Eng Biotechnol 1007; 104:1-11, which is incorporated by reference.

For example, a method for determining the methylation state of nucleic acids is described in U.S. Pat. No. 6,017,704 which is incorporated by reference. Determining the methylation state of the nucleic acid includes amplifying the nucleic acid by means of oligonucleotide primers that distinguishes between methylated and unmethylated nucleic acids.

Methylation specific PCR (MSP) is disclosed in U.S. Pat. Nos. 5,786,146, 6,200,756, 6,017,704 and 6,265,171, each of which is incorporated by reference. Furthermore, a combination of DNA markers for CpG-rich regions of nucleic acid may be amplified in a single amplification reaction. The markers are multiplexed in a single amplification reaction, for example, by combining primers for more than one locus. In one embodiment, DNA from a normal tissue surrounding a polyp can be amplified with two or more different unlabeled or randomly labeled primer sets in the same amplification reaction. The reaction products can be separated on, for example, a denaturing polyacrylamide gel and subsequently exposed to film or stained with ethidium bromide for visualization and analysis.

By analyzing a panel of genes, there may be a greater probability of producing a more useful methylation profile for a subject. Multigene MSP may employ MSP primers for a plurality of markers, for example up to two, three, four, five or more different colorectal cancer marker, in a two-stage nested PCR amplification reaction. As in typical two stage primer PCR reactions, the primers used in the first PCR reaction are selected to amplify a larger portion of the target sequence than the primers of the second PCR reaction. The primers used in the first PCR reaction are generally referred to the DNA primers and the primers used in the second PCR reaction are the MSP primers. MSP primers generally comprise two sets of primers: methylated and unmethylated for each of the markers that are being assayed. Methods of multigene MSP are disclosed in U.S. Pat. No. 6,835,541, which is incorporated by reference.

Detection of differential methylation can also be accomplished by contacting a nucleic acid sample with methylation-sensitive restriction endonucleases that cleave only unmethylated CpG sites under appropriate conditions and for an appropriate length of time to allow cleavage of unmethylated nucleic acid. The sample can also be contacted with isoschizomers of the methylation-sensitive restriction endonucleases that cleave both methylated and unmethylated CpG-sites under appropriate conditions and for an appropriate length of time to allow cleavage of methylated nucleic acid. Oligonucleotides are subsequently added to the nucleic acid sample under appropriate conditions and for an appropriate length of time to allow ligation of the added oligonucleotides to the cleaved nucleic acid. The ligated composition of nucleic acid from sample and oliogonucleotides can then be amplified by conventional methods, such as PCR, where the primers are complementary to the added oligonucleotides.

“Methylation-sensitive restriction endonuclease” are well known in the art and are generally considered to be is a restriction endonuclease that includes CG as part of its recognition site and has altered activity when the C is methylated as compared to when the C is not methylated. In one embodiment, the methylation-sensitive restriction endonuclease has inhibited activity when the C is methylated (e.g., Smal). Examples of methylation-sensitive restriction endonucleases include, but are not limited to, Sma I, BssHII, or HpaII, MspI, BSTUI, SacII, EagI, and NotI. Of course, these enzymes can be used alone or in combination with other enzymes. As used herein, an “isoschizomer” of a methylation-sensitive restriction endonuclease is a restriction endonuclease that recognizes the same recognition site as a methylation sensitive restriction endonuclease but cleaves both methylated and unmethylated CGs. Those of skill in the art can readily determine appropriate conditions for a restriction endonuclease to cleave a nucleic acid (see Sambrook et al., Molecular Cloning: A Laboratory Manual, Cold Spring Harbor Press, 1989).

The measure of the levels of methylation may contain a qualitative component, or it may be quantitative. For example, the methylation status of a gene or panel of genes may simply be considered, on the whole, as methylated or unmethylated, or the methylation status may be quantified as some numerical expression, such as a ratio or a percentage. Furthermore, the methylation status of each individual member of the gene or panel of genes may be assessed, or the methylation status of the gene or panel of genes, as a whole, may be assayed, determined or considered.

The methylation status of the subject may be assessed in vivo or in vitro, from a sample from the subject. The samples may or may not have been removed from their native environment. Thus, the portion of sample assayed need not be separated or removed from the rest of the sample or from a subject that may contain the sample. Of course, the sample may also be removed from its native environment. For example, the sample may be a tissue section. The tissue section may be, for example, a portion of the neoplasm that is being treated or it may be a portion of the surrounding normal tissue. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

Once the methylation status and level of the gene or panel of genes have been determined, these determinations can then be used to predict, indicate, or otherwise assess or predict the likelihood the abnormal condition, e.g., a polyp, will already be present, develop in the future, or recur in the future in the patient. As used herein, a subject in which the “condition recurred,” i.e., a progressor subject, is used to indicate that the abnormal condition recurred in the subject after successful ablative treatment. As used herein, “predict” means to provide an indicia of whether a particular abnormal condition will recur after treatment or if the abnormal condition will develop in subject. As used herein, indicate means to provide a basis to a health care practitioner whether a particular condition will recur in the subject.

To predict the development or recurrence of the abnormal condition, the methylation status or level of the test subject's gene or panel of genes may be compared to one or more progressor subjects, including, but not limited to a population of progressor subjects. Or the methylation status or level of the test subject's gene or panel of genes may be compared to one or more non-progressor subjects, including, but not limited to a population of non-progressor subjects. In addition, the methylation status or level of the gene or panel of genes in the test subject may be compared to his or her own previously assessed methylation status of the gene or panel of genes. In another embodiment, the methylation status or level of the gene or panel of genes in the test subject is compared to a normal methylation status or level of the gene or panel of genes.

“Normal methylation status or level” may be assessed by measuring the methylation status or level in a known healthy subject, including the same subject that is later screened or being diagnosed. Normal levels may also be assessed over a population of samples, where a population sample is intended to mean either multiple samples from a single subject or at least one sample from a multitude of subjects. Normal methylation levels of the gene or panel of genes, in terms of a population of samples, may or may not be categorized according to characteristics of the population including, but not limited to, sex, age, weight, ethnicity, geographic location, fasting state, state of pregnancy or post-pregnancy, menstrual cycle, general health of the subject, alcohol or drug consumption, caffeine or nicotine intake and circadian rhythms.

It will be appreciated by those of skill in the art that a baseline or normal level need not be established for each assay as the assay is performed but rather, baseline or normal levels can be established by referring to a form of stored information regarding a previously determined baseline methylation levels for a given gene or panel of genes, such as a baseline level established by any of the above-described methods. Such a form of stored information can include, for example, but is not limited to, a reference chart, listing or electronic file of population or individual data regarding “normal levels” (negative control) or polyp positive (including staged tumors) levels; a medical chart for the patient recording data from previous evaluations; a receiver-operator characteristic (ROC) curve; or any other source of data regarding baseline methylation levels that is useful for the patient to be diagnosed.

Further a methylation index (MI) may be established. A methylation index (MI) is defined as the number of genes which demonstrated altered methylation status (i.e., which exceed or fall below a previously determined methylation level cutoff) within a defined set of genes. For example, if there are four genes in a defined gene set and none of these four genes is methylated, the MI equals 0; if any one of the four are methylated, the MI equals 1; if any two of the four are methylated, the MI equals 2; if any three of the four are methylated, the MI equals 3; and if all four of these four genes are methylated, the MI equals 4 (i.e., the maximum possible MI for this gene set).

The difference between the methylation status or level of the test subject and normal methylation levels may be a relative or absolute quantity. Thus, “methylation level” or “methylation status” is used to connote any measure of the quantity of methylation of the gene or panel of genes. The level of methylation may be either abnormally high, or abnormally low, relative to a defined high or low threshold determined to be normal for a particular group of subjects. The difference in level of methylation between a subject and the reference methylation level may be equal to zero, indicating that the subject is or may be normal, or that there has been no change in levels of methylation since the previous assay.

The methylation levels and any differences that can be detected may simply be, for example, a measured fluorescent value, radiometric value, densitometric value, mass value etc., without any additional measurements or manipulations. Alternatively, the levels or differences may be expressed as a percentage or ratio of the measured value of the methylation levels to a measured value of another compound including, but not limited to, a standard or internal DNA standard, such as beta-actin. This percentage or ratio may be abnormally low, i.e., falling below a previously defined normal threshold methylation level; or this percentage or ratio may be abnormally high, i.e., exceeding a previously defined normal threshold methylation level. The difference may be negative, indicating a decrease in the amount of measured levels over normal value or from a previous measurement, and the difference may be positive, indicating an increase in the amount of measured methylation levels over normal values or from a previous measurement. The difference may also be expressed as a difference or ratio of the methylation levels to itself, measured at a different point in time. The difference may also be determined using in an algorithm, wherein the raw data is manipulated.

A difference between the test subject's methylation status between two time points is an indication that the test subject may or may have an increased likelihood of concurrent presence, future occurrence, or future recurrence of the abnormal condition in the subject. For example, a methylation status in the test subject at a first time point that is greater than the methylation status of the test subject at a second time point may indicate that there may be a lower likelihood of the concurrence, future occurrence, or recurrence of the abnormal condition in the subject, whereas the abnormal condition at time point one was predicted to be present, occur, or recur after treatment. Alternatively, a methylation status in the test subject that is lower at a first time point than the methylation status in the test subject at a second time point may indicate that the there is an increased likelihood that the abnormal condition will be present, occur, or recur in the subject, from the first time point. An inverse relationship, however, may also exist between the methylation status of the gene or panel of genes (or the difference thereof) and the subject's likelihood for an abnormal condition being present, developing in the future, or recurring in the future.

The present invention also provides methods of customizing a therapeutic regimen for a subject in need thereof, with the methods comprising determining the methylation status or level of a gene or panel of genes in a test subject and using the methylation status or level of the test subject to dictate an appropriate therapeutic regimen going forward or indicate the responsiveness of a particular therapeutic regimen going forward.

The present invention also provides methods of monitoring the progression of an abnormal condition in a subject, with the methods comprising determining the methylation status or level of a gene or panel of genes in a test subject at a first and second time point to determine a difference in methylation status or level of the gene or panel of genes in the subject over time. A difference in methylation status in the gene or panel of genes in the subject over time may be indicative of the occurrence, recurrence, or progression of the abnormal condition.

As used herein, the phrase “monitor the progression” is used to indicate that the abnormal condition in the subject is being periodically checked to determine if the abnormal condition is progression (worsening), regressing (improving), or remaining static (no detectable change) in the individual by assaying the methylation status or level in the subject using the methods of the present invention. The methods of monitoring may be used in conjunction with other monitoring methods or other treatments for the abnormal condition to monitor the efficacy of the treatment. Thus, “monitor the progression” is also intended to indicate assessing the efficacy of a treatment regimen by periodically assessing the methylation status of the gene or panel of genes and correlating any differences in methylation status in the subject over time with the progression, regression or stasis of the abnormal condition. Monitoring may include two time points from which a sample is taken, or it may include more time points, where any of the methylation status or level data at one particular time point from a given subject may be compared with the methylation status or level data in the same subject, respectively, at one or more other time points.

The present invention also provides methods of diagnosing a disease state in a subject suspected of having a disease, with the methods comprising determining the methylation status or level of a gene or panel of genes in a test subject and using the test subject's methylation status or level to indicate the presence of a disease state in the subject.

As used herein, the term “diagnose” means to confirm the results of other tests or to simply confirm suspicions that the subject may have an abnormal condition, such as cancer. A “test,” on the other hand, is used to indicate a screening method where the patient or the healthcare provider has no indication that the patient may, in fact, have an abnormal condition and may also be used to assess a patient's likelihood or probability of developing a disease or condition in the future. The methods of the present invention, therefore, may be used for diagnostic or screening purposes. Both diagnostic and testing can be used to “stage” the abnormal condition in a patient. As used herein, the term “stage” is used to indicate that the abnormal condition or obesity can be categorized, either arbitrarily or rationally, into distinct degrees of severity. The term “stage,” however, may or may not involve disease progression. The categorization may be based upon any quantitative characteristic or be based upon qualitative characteristics that can be separated. An example of staging includes but is not limited to the Tumor, Node, Metastasis System of the American Joint Committee on Cancer. For example, in stage T1 of colorectal cancer, the tumor has grown through the muscularis mucosa of the colon and extends into the submucosa. In stage T2, the cancer has grown through the submucosa, and extends into the muscularis propria. In stage T3, the cancer has grown completely through the muscularis propria into the subserosa, but not to any neighboring organs or tissues. And in stage T4, the cancer has spread completely through the wall of the colon or rectum into nearby tissues or organs. Other examples of staging systems include, but are not limited to, the Dukes system and the Astler-Coller system.

In one particular embodiment of the diagnostic methods, the present invention provides methods of assessing the probability of a subject having an abnormal condition, with the methods comprising determining a methylation status or level of at least one gene in grossly normal tissue of the subject and comparing the methylation status or level of the gene or genes in said subject to the normal methylation status or level of the at least one gene. As used herein, grossly normal tissue is used to indicate that the tissue from which the sample is taken appears normal upon gross inspection (i.e., by the naked eye). In other words, a technician or clinician who removes a sample or biopsy from the subject may remove the sample from what appears to be normal tissue. Once the grossly normal tissue is removed, DNA from the cells of the grossly normal tissue is isolated and the methylation status or level of a gene or panel of genes is determined in the cells' DNA that has been taken from the grossly normal tissue. The methylation status or level of the gene or panel of genes from the grossly normal tissue from the subject is then compared to the normal methylation status or level of the same gene or panel of genes to determine if any difference exists between the subject's status or level and previously defined normal status or level. A difference between the subject's methylation status or level and the normal methylation status or level of the gene or panel of genes indicates that the subject may have an altered probability of having or developing an abnormal condition elsewhere in the body. For example, the methylation status or level of a subject's rectum that is normal upon gross inspection can be compared to accepted normal methylation status or level. If a difference exists between the subject's methylation status or level in grossly normal rectum and the previously defined normal methylation status or level, this difference indicates that the subject may currently have, or develop in the future, an abnormal condition elsewhere in the remaining portion of the colon. These abnormal conditions that may be screened using grossly normal tissue from subjects include, but are not limited to, the abnormal conditions described herein.

The present invention also provides for kits for performing the methods described herein. Kits of the invention may comprise one or more containers containing one or more reagents useful in the practice of the present invention. Kits of the invention may comprise containers containing one or more buffers or buffer salts useful for practicing the methods of the invention. A kit of the invention may comprise a container containing a substrate for an enzyme, a set of primers and reagents for PCR, etc.

Kits of the invention may comprise one or more computer programs that may be used in practicing the methods of the invention. For example, a computer program may be provided that calculates a methylation status in a sample from results of the detecting levels of antibody bound to the biomarker gene product of interest. Such a computer program may be compatible with commercially available equipment, for example, with commercially available microarray or real-time PCR. Programs of the invention may take the output from microplate reader or realtime-PCR gels or readouts and prepare a calibration curve from the optical density observed in the wells, capillaries, or gels and compare these densitometric or other quantitative readings to the optical density or other quantitative readings in wells, capillaries, or gels with test samples.

The examples are meant to be illustrative in nature and should not limit the scope of the invention described herein.

### EXAMPLE 1

### Patient and Sample Selection

Fifty-six primary human colon cancers, 22 non-cancerous human colon mucosae, and 14 human colon cancer cell lines were selected for study. Colonic tissues were macroscopically dissected to enrich colonic mucosal layers or tumor cells. Genomic DNAs and total RNAs were extracted from snap-frozen tissues. Tumor microsatellite instability (MSI) status was determined based upon 11 microsatellite markers as described previously as previously described (Mori, Y. et al., Cancer Res., 63:4577-4582 (2003), which is incorporated by reference). Briefly, specimens with microsatellite instability (MSI) at 30% or more of informative loci were labeled as microsatellite-unstable (MSI-H), specimens showing MSI at less than 30% of informative loci were labeled as having low-level microsatellite instability (MSI-L), and specimens with no MSI were labeled as microsatellite stable (MSS). All colonic tissues were based upon the availability of a sufficient amount of high quality nucleic acids (DNA and/or RNA), clinicopathological data, and informative MSI data at more than five microsatellite loci including BAT25 and BAT26.

Gene Selection

Initially, a cDNA microarray analysis was performed comparing seven normal colonic mucosae versus 12 MSI-Hor 15 MSS primary colon cancers. The cDNA microarray analysis utilized an in-house 8,064 human gene microarray. In brief, 30 μg of total RNA were amplified using a T7-based protocol, and 6 μg of the resulting amplified RNA (aRNA) were labeled with Cy5-dCTP. An aRNA pool of human cancer cell lines was used as the reference probe and labeled with Cy3-dCTP. The expression level of each gene was represented by the log-transformed Cy5-Cy3 intensity ratio, and global intensity-based normalization was performed. To identify genes that were downregulated in colon cancers relative to normal colonic mucosae, a Significance Analysis of Microarray (SAM) was performed to compare seven normal colonic mucosae to 12 MSI-H colon cancers or to 15 MSS colon cancers. A gene was excluded from SAM analysis when it lacked expression data in more than 30% of the normal tissue group or the tumor group. Differentially expressed genes were determined at a false discovery rate (FDR) of less than 12%. 1,874 genes were downregulated in either MSI-H- or MSS-cancers relative to normal colonic mucosae.

Next, a cDNA microarray analysis of two colon cancer cell lines was performed, comparing cells treated with 5-aza-dC and untreated cells. Briefly, the colon cancer cell lines HT29 and HCT116 were seeded at a density of 4×10⁵ cells per 75 cm² culture flask and incubated for 24 hours in growth media. The cells were then treated with 5-aza-dC in either one of the following conditions: a) 10 μM 5-aza-dC for 84 hours or b) 1 μM 5-aza-dC for 24 hours followed by 60 hours of 5-aza-dC-free growth. These two 5-aza-dC treatment regimens were employed to obtain expression data under diverse cellular conditions: the former regimen focused on maximum DNA demethylation, while the latter regimen focused on moderate DNA demethylation to minimize damage to cell viability. The log-transformed ratio of the expression level for the pre-treatment specimen relative to that of the post-treatment specimen was calculated to identify genes that were upregulated by 5-aza-dC treatment in HCT116 and/or HT29 cells. A gene was classified as upregulated by 5-aza-dC treatment when this value was greater than 0.75 (equivalent to 1.65-fold up-regulation) in at least one treatment condition in at least one cell line. Genes with very low expression levels in post-treatment specimens were eliminated from further analyses. The cut-off criterion was defined as median signal intensity less than median background intensity plus 1 standard deviation.

Over eight hundred genes (861) genes were upregulated in at least one of these cell lines after 5-aza-dC treatment. By combining these two microarray analyses, we identified 274 genes with mRNA expression patterns that were appropriate for tumor-specific promoter methylation target genes. These 274 genes were downregulated in primary colon cancers, and, simultaneously, these 274 genes were upregulated in colon cancer cells after global DNA demethylation.

Next, we performed an in silico search to determine the identity of some of the candidate genes. The presence of a CpG island overlapping the 5′ untranscribed region (5′UTR) or first exon of a target gene was examined using the USCS Human Genome Browser database available on the world wide web at www.genome.ucsc.edu/ceibin/hgGatewav according to their CpG island definition. The presence of a putative promoter within target CpG islands was verified using WWW Promoter Scan (bimas.dcrt.nih.gov:80/molbio/proscan/) and the Eukaryotic Promoter Database, also available on the world wide web at www.epd.isb-sib.ch/index.html. The putative function of a target gene was obtained using SWISS-Prot (au.expasy.org/sprot), Entrez Gene (www.ncbi.nlm.nih.gov/entrez/query.fcgi?db=gene), and FatiGO database (www.fatigo.org). The in silico search revealed that 214 of these 274 genes were autosomal genes with CpG islands overlapping their putative promoter regions.

Furthermore, 54 within this 214-gene subset possessed functions that were potentially relevant to cancer. Of the 54 genes identified in above, three genes, RUNX3, HLTF/SMARCA3, and RAB32, had been extensively studied before, were excluded from further study. Promoter region methylation status of the remaining 51 novel candidates was assessed in 14 colon cancer cell lines using MSP. Nine of these 51 genes demonstrated methylation in at least one of the 14 cell lines. These 9 genes were further analyzed in 34 primary colon cancers and 17 normal colonic mucosae for promoter region methylation status.

DNA Treatment and Methylation-Specific PCR

Tumor samples were snap frozen on dry ice and stored at −80° C. After thawing, DNA was extracted from samples and treated with bisulfite prior to MSP. Briefly, DNA was extracted from all samples and treated with bisulfite to convert unmethylated cytosines to uracils prior to methylation-specific PCR (MSP) as described previously in Mori, Y., et al. Cancer Res. 64:2434-38 (2004), which is incorporated by reference. DNA methylation status and levels of the 4 candidate markers were determined with real-time quantitative MSP using the ABI 7900 HT Sequence Detection (Taqman) System, as described previously in Sato F., et al., Cancer Res. 62:6820-22 (2002), which is incorporated by reference. Primers and probes for quantitative MSP of (SHP-1, DcR1, RARβ, and DcR2) are disclosed in Takahashi, T. et al, Int'l J. Cancer, 118(4): 924-931 (2005), which is incorporated by reference.

The Sodium Bisulfite Conversion of DNA was performed using the EpiTect BiSulfite Kit, available from Qiagen, according to the manufacturer's suggested protocol. Briefly, DNA was thawed and dissolve by adding 800 μl RNase-free water to each aliquot. The dissolved DNA was vortexed until the Bisulfite Mix was completely dissolved. On occasion, it was necessary to heat the water/DNA mixture to about 60° C. to aid in dissolving of the DNA. Bisulfite reactions were prepared in 200 μl PCR tubes according to Table I (each component was added in the order listed).

TABLE I
Bisulfite Reaction Components

| Component | Volume per Reaction (μL) |
|---|---|
| DNA solution (1 ng-2 μg) | Variable*(maximum 20) |
| RNase-free water | Variable* |
| Bisulfite Mix (dissolved) | 85 |
| DNA Protect Buffer | 35 |
| Total volume | 140 |

*The combined volume of DNA solution and RNase-free water must total 20 μl.

After mixing, the PCR tubes are stored at room temperature. Next, the bisulfite DNA conversion was performed using a thermal cycler that was programmed according to the parameters in Table II.

TABLE II
Bisulfite Conversion Thermal Cycler Conditions

| | | |
|---|---|---|
| Step | Time | Temperature |

| | | | |
|---|---|---|---|
| Denaturation | 5 | Min | 99° C. |
| Incubation | 25 | Min | 60° C. |
| Denaturation | 5 | Min | 99° C. |
| Incubation | 85 | Min | 60° C. |
| Denaturation | 5 | Min | 99° C. |
| Incubation | 175 | Min | 60° C. |

| | | |
|---|---|---|
| Hold | Indefinite | 20° C. |

Once the bisulfite conversion was complete, the PCR tubes were centrifuged and transferred to clean 1.5 ml microcentrifuge tubes. 560 μl of freshly prepared Buffer BL (containing 10 μg/ml carrier RNA) was then added and mixed by vortexing and centrifugation. The EpiTect spin columns were placed in a and collection tube in a suitable rack and the mixture was transferred into the EpiTect spin column. The columns were centrifuged at maximum speed for about 1 minute and the flow-through was discarded. The spin columns were placed back into the collection tubes and 500 μl Buffer BW (wash buffer) was to the spin columns. Again, the spin columns were centrifuged at maximum speed for about 1 minute, and the flow-through was discarded. The spin columns were placed back into the collection tubes.

Next, 500 μl of Buffer BD (desulfonation buffer) was added to each spin column, and the columns were incubated for about 15 minutes at room temperature. After incubation, the columns were centrifuged at maximum speed for about 1 minute. The flow-through was discarded, and the columns were placed back into the collection tubes.

500 μl Buffer BW was added to the columns and the columns were centrifuged at maximum speed for about 1 min. The flow-through was discarded, and the spin columns were placed back into the collection tube. This washing step was repeated at lease one more time.

After repeated washing, the spin columns were placed into new 2 ml collection tube, and the columns were centrifuged at maximum speed for about 1 to 5 minutes to remove any residual liquids. Finally, the spin columns were placed into clean 1.5 ml microcentrifuge tubes and 20 μl of Buffer EB was to the center of the membrane in the spin column. The purified DNA was then eluted by centrifugation for about 1 minute at approximately 15,000×g (12,000 rpm).

DNA methylation status and levels of 5 genes were determined with real-time quantitative MSP using the ABI 7900 HT Sequence Detection (Taqman) System, as described previously in Sato F., et al., Cancer Res. 62:6820-22 (2002), which is incorporated by reference. Primers and probes for quantitative MSP of ENG, MAL, CAV1, TAC1, SST, NELL1 and AKAP12 are below in Tables III and IV.

TABLE III
Forward and Reverse Primers

| | | |
|---|---|---|
| | Forward Primer | Reverse Primer |
| Gene | sequence (5′>3′) | sequence (5′>3′) |

| | | |
|---|---|---|
| ENG | AGGGTTTTTTATTTAG | CTAACTCATCCAACCCG |
| | TGATAAAGTTCGTGG | ACCG |
| MAL | AGTTTTTAGTTTTGGA | CGAAAAACCCGACCCGA |
| | CGTTCGTAGCG | ACG |
| CAV1 | CGAAGCGTTTGGGAGA | ATACTTTTAATAACACT |
| | TATTTTAGAAAC | CGTTTACATCTAATCG |
| TAC1 | GGCGGTTAATTAAATA | AAATCCGAACGCGCTCT |
| | TTGAGTAGAAAGTCGC | TTCG |
| SST | GGGGCGTTTTTTAGTT | AACAACGATAACTCCGA |
| | TGACGT | ACCTCG |
| NELL1 | GGGTTTTTAGACGAAC | CCACCGCTAACGCGACG |
| | GTAGTCGTAGC | A |
| AKAP12 | GTTTTGTTAGAAACGG | GAAACCAAAAACGCTAC |
| | GAGGCGTTC | GACGCG |

TABLE IV
Methylation-Specific PCR Probes

| | |
|---|---|
| Gene | (5′>3′) |

| | |
|---|---|
| ENG | TCGGTTGGTAGGCGGTTTGGTTTAGT |
| MAL | TCGAAGAGGTTTAGGGCGGTGTTCG |
| CAV1 | AAACTTAAACAAACATACAAAATTTAACATTTCCCATC |
| TAC1 | AGAATGTTACGTGGGTTTGGAGGTT |
| SST | AACGACTTATATACTCTCAACCGTCTCCCTCTA |
| NELL1 | ACGACGTAAAACTCGAAACCCGACCC |
| AKAP12 | TCGGGTGGGCGGTTGTTTGGATT |
| ENG | AGACCAGGAAGTCCATAGGACTGTCTTCAT |

A normalized methylation value (NMV) reflecting the percentage of DNA methylated for the gene of interest (GoI), was defined as follows: NMV=(GoI-S/GoI-FM)/(ACTB-S/ACTB-FM)*100, where GoI-S and GoI-FM represented GoI methylation levels in the Sample and Fully Methylated DNAs, respectively, while ACTB-S and ACTB-FM corresponds to β-Actin in the sample and Fully Methylated (FM) DNAs, respectively.

Statistical Analysis

P-values were calculated using Mann-Whitney's U-test or Fisher's exact test to assess the associations between MI and various clinico-pathological characteristics. The association between age and MI in primary cancers was assessed using multiple regression analysis, as well as Mann-Whitney's U-test. For MannWhitney's U-test, four different cut-off ages (60, 65, 70, 75) were applied to divide the cancers into two groups based upon their age.

Markers for Diagnosis of Colon Cancer

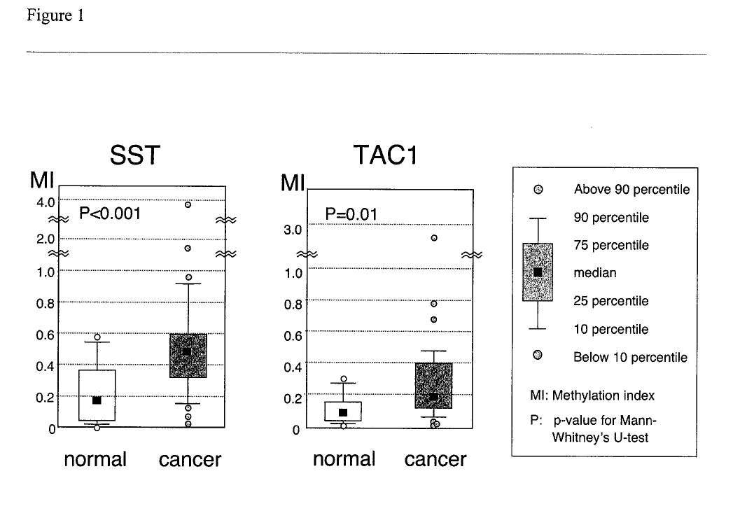

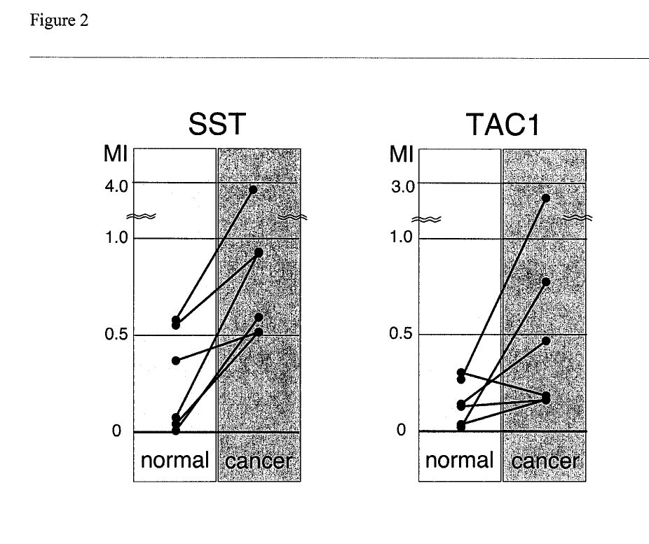

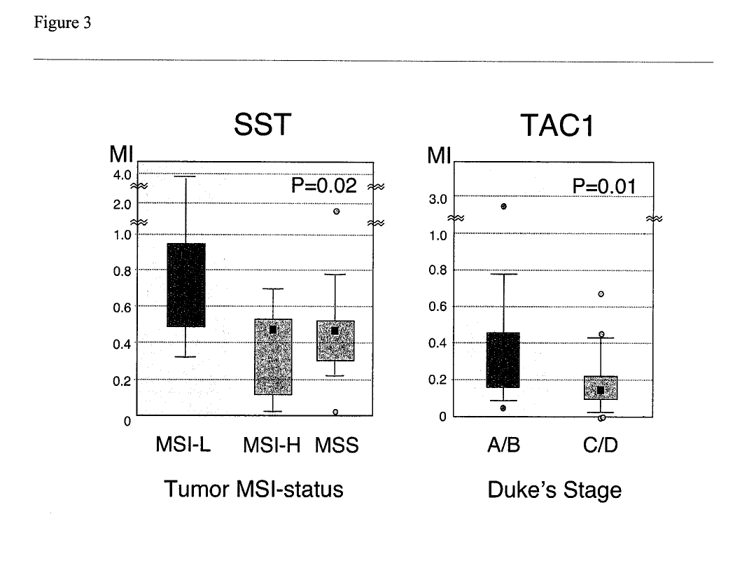

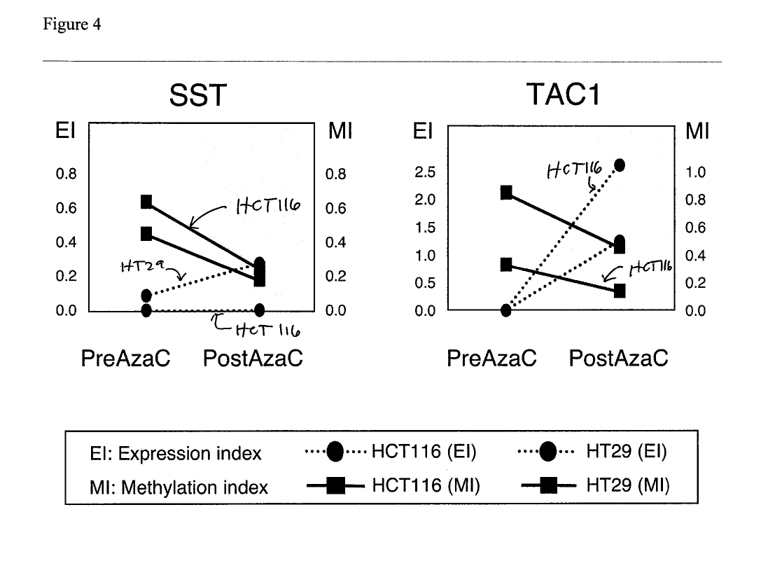

As shown in FIG. 1, both SST (30 of 34 cases, 88%) and TAC1 (16 of 34 cases, 47%) were frequently methylated in primary colon cancers. Normal colonic mucosae also demonstrated some degree of methylation (SST, 8 of 17 cases, 47%; TACT, 2 of 17 cases, 12%). The methylation levels of SST and TAC1, however, were significantly lower in normal colonic mucosae than in colon cancers (p<0.001 and p=0.01, respectively, Mann-Whitney U-test). Increased SST and TAC1 methylation levels was also observed in cancers relative to matched normal colonic mucosae from the resection margins (FIG. 2). TAC1 methylation levels were significantly higher in Duke's stage A/B cancers than in Duke's stage C/D cancers (p=0.01, Mann-Whitney's U-test; FIG. 3). Similarly, SST methylation levels were significantly higher in MSI-L cancers than in MSS and MSI-H cancers (p=0.02, Mann-Whitney's U-test; FIG. 3). Age, gender, tumor site or histological differentiation were not significantly associated with methylation level of SST or TACT (data not shown). Furthermore, we examined the correlation between promoter methylation and mRNA expression levels of SST and TAC1. For this purpose, we analyzed colon cancer cell lines with and without 5-aza-dC treatment using quantitative real-time PCR. mRNA upregulation occurred for both SST and TAC1 after promoter DNA demethylation by 5-aza-dC treatment (FIG. 4).

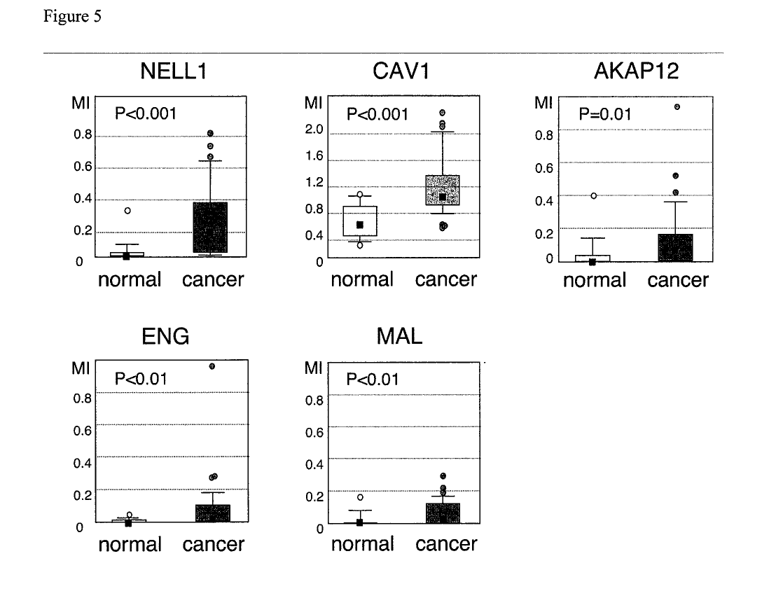

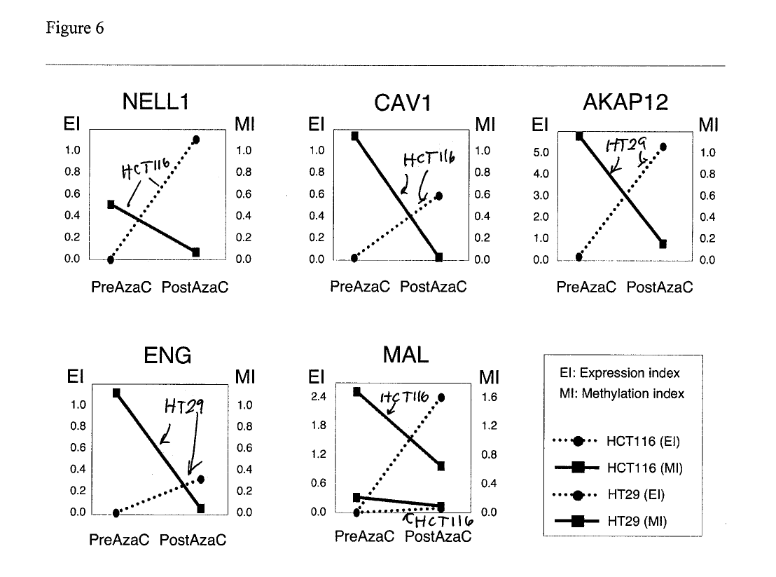

In addition to SST and TACT, hypermethylation of five other genes were detected in primary colon cancers by our epigenetic analysis of primary colon cancers and normal colonic mucosae: the protein kinase C binding protein gene NELL1 (15/34, 44%), the scaffold protein gene caveolin-1 (CAV1, 34/34, 100%), the a-kinase anchoring protein gene AKAP12 (7/34, 21%), the TGF-beta receptor gene endoglin (ENG, 3/34, 9%), and the T-cell differentiation protein gene MAL (2/34, 6%) (FIG. 5). Methylation of these genes in normal colonic mucosae was absent (ENG and MAL,) or significantly less frequent than in primary cancers (AKAP12, CAV1, and NELL1; p=0.01, p<p. 001, and p<0.001, respectively, Mann-Whitney U-test). Age was not significantly associated with methylation levels in primary colon cancers in any of these genes (data not shown). For all five of these genes, mRNA was increased after promoter region DNA demethylation by 5-aza-dC treatment in a colon cancer cell line (FIG. 6).

### EXAMPLE 2

Patient and Sample Selection

67 normal esophagi (NE), 60 Barrett's metaplasias without dysplasia (BE, including 36 obtained from patients with Barrett's only (Ba) and 24 from patients with Barrett's accompanied by EAC (Bt)), 40 dysplasias occurring in BE (D, including 19 low-grade (LGD) and 21 high-grade (HGD)), 67 EACs, and 24 ESCCs were examined. Biopsies were obtained using a standardized biopsy protocol, as previously described. Research tissues were taken from grossly apparent Barrett's epithelium or from mass lesions in patients manifesting these changes at endoscopic examination, and histology was confirmed using parallel immediately adjacent aliquots obtained at endoscopy. All specimens were stored in liquid nitrogen before DNA extraction. In addition, three EAC esophageal adenocarcinoma (BIC, OE33 and SEG) and nine ESCC esophageal squamous cell carcinoma (KYSE 110, 140, 180, 200, 220, 410, 450, 520 and 850) cell lines were examined. These cells were cultured in 47.5% RPMI 1640, 47.5% F-12 supplemented with 5% fetal bovine serum.

Sample Treatment

Samples and cell lines were treated as in Example 1. Briefly, extracted DNA was treated with bisulfite to convert unmethylated cytosines to uracils prior to MSP. Promoter methylation levels of TAC1 were determined using qMSP with the ABI 7700 Sequence Detection (Taqman) System, using primers and probes described. A normalized methylation value (NMV), reflecting the percentage of DNA methylated for the gene of interest (GoI), was defined as follows: NMV=(GoI-S/GoI-FM)/(ACTB-S/ACTB-FM), where GoI-S and GoI-FM represent GoI methylation levels in the sample and fully methylated DNAs, respectively, while ACTB-S and ACTB-FM correspond to β-Actin in the sample and fully methylated DNAs, respectively.

To determine TAC1 mRNA levels, one-step real-time quantitative RT-PCR was performed using a Qiagen QuantiTect Probe RT-PCR Kit (Qiagen, Hilden, Germany) and the ABI 7700 Sequence Detection (Taqman) System. Primers and probes are described herein. β-Actin was used to normalize data. A standard curve was generated using serial dilutions of qPCR Reference Total RNA (Clontech, Mountainview, Calif.). The normalized mRNA value (NRV) was calculated according to the following formula for relative expression of target mRNA: Expression index=(TarS/TarC)/(ACTB-S/ACTB-C, where TarS and TarC represent levels of mRNA expression for the target gene in the sample and control mRNAs, respectively, whereas ACTB-S and ACTB-C correspond to amplified ACTB levels in the sample and control mRNAs, respectively.

To determine SST mRNA levels, one-step real-time quantitative RT-PCR was performed using a Qiagen QuantiTect Probe RT-PCR Kit (Qiagen, Hilden, Germany) and the ABI 7700 Sequence Detection (Taqman) System. Primers and probes were the same as previously reported. 12 β-Actin was used for normalization of data. A standard curve was generated using serial dilutions of qPCR Reference Total RNA (Clontech, Mountainview, Calif.). The normalized mRNA value (NRV) was calculated according to the following formula for relative expression of target mRNA: NRV=(TarS/TarC)/(ACTB-S/ACTB-C), where TarS and TarC represent levels of mRNA expression for the target gene in sample and control mRNAs, respectively, whereas ACTB-S and ACTB-C correspond to amplified ACTB levels in sample and control mRNAs, respectively.

To determine whether TAC1 inactivation was due to promoter hypermethylation in esophageal cancer, 2 esophageal cancer cell lines (KYSE220 and BIC) were subjected to 5-Aza-dC (Sigma, St. Louis, Mo.) treatment. Briefly, 1×10⁵ cells/ml were seeded onto a 100 mm dish and grown for 24 h. Then, 1 μl of 5 mM 5-Aza-dC per ml of cells was added every 24 hours for 6 days. DNAs and RNAs were harvested on day 6.

To determine whether SST inactivation was due to promoter hypermethylation in esophageal cancer, 2 esophageal cancer cell lines (KYSE220 and OE33) were subjected to 5-Aza-dC (Sigma, St. Louis, Mo.) treatment. Briefly, 1×10⁵ cells/ml were seeded onto a 100 mm dish and grown for 24 h. Then, 1 μl of 5 mM 5-Aza-dC per ml of cells was added every 24 hours for 6 days. DNA and RNA were harvested on day 6.

Statistical Analysis

Receiver-operator characteristic (ROC) curve analysis was performed using NMVs for the 67 EAC, 24 ESCC and 67 NE by Analyse-it© software (Version 1.71, Analyse-it Software, Leeds, UK). Using this approach, the area under the ROC curve (AUROC) identified optimal sensitivity and specificity levels at which to distinguish normal from malignant esophageal tissues, and corresponding NMV thresholds were calculated for TAC1. The cutoff value determined from this ROC curve was applied to determine the frequency of TAC1 methylation in each tissue type included in the present study. For all other tests, Statistica (version 6.1; StatSoft, Inc., Tulsa, Okla.) was used. Differences with p<0.05 were deemed significant.

Markers for Diagnosis of Esophogeal Squamous Cell Carcinoma

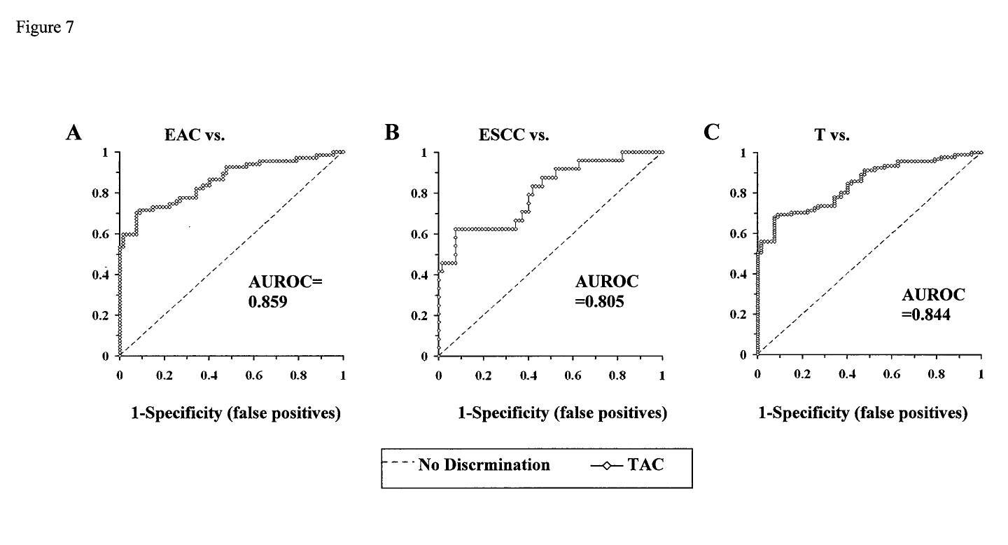

TAC1 promoter hypermethylation was analyzed in 24 ESCC and 67 EAC, 40 D (19 LGD and 21 HGD), 60 BE (36 Ba and 24 Bt), and 67 NE. TAC1 promoter hypermethylation showed highly discriminative ROC curve profiles, which clearly distinguished both ESCC (p<0.0001) and EAC (p<0.0001) from NE. ROC curves with corresponding areas under the ROC curve (AUROCs) for TAC1 of ESCC vs. NE, EAC vs. NE, and combined esophageal cancer (T) vs. NE are shown in FIG. 7.

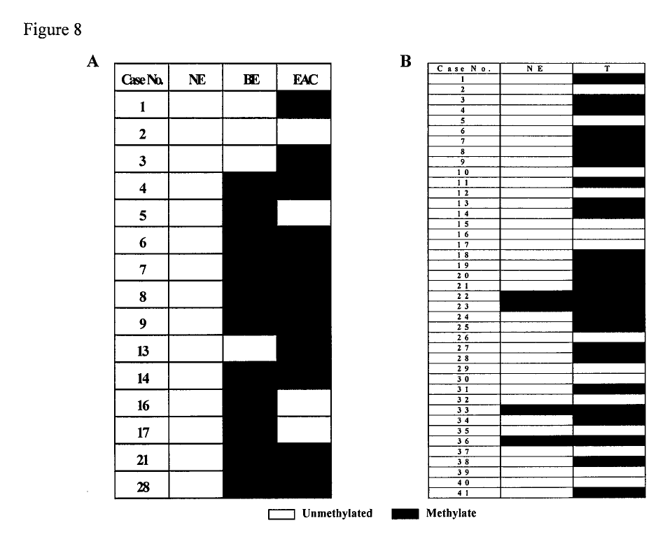

The cutoff NMV for TAC1 (0.12) was chosen from the ROC curve to maximize sensitivity and specificity. The NMV of TAC1 was significantly higher in ESCC, EAC, D, HGD, LGD, Ba, Bt and BE than in NE (p<0.0000001, Student's t-test). Similarly, increased frequencies of TAC1 hypermethylation were observed in Ba (55.6%), D (57.5%), and EAC (61.2%) relative to NE (7.5%). Both TAC1 hypermethylation frequency and mean NMV were higher in Bt than in Ba (75% vs. 55.6% and 0.2313 vs. 0.2145, respectively). Among 15 cases with corresponding NE, BE and EAC, one (No. 2) was unmethylated, two (No. 3 and 13) were methylated only in EAC, three (No. 5, 16 and 17) were methylated only in BE, and the remaining nine were methylated in both BE and EAC (FIG. 8A). Among 41 cases with corresponding NE and T, four (100%) of four cases (No. 22, 23, 33 and 36) showing methylation in NE were also methylated in corresponding EAC (FIG. 8B), and the TAC1 NMVs of T (mean=0.209) were significantly higher than those of corresponding NE (mean=0.056) (p<0.0000001,

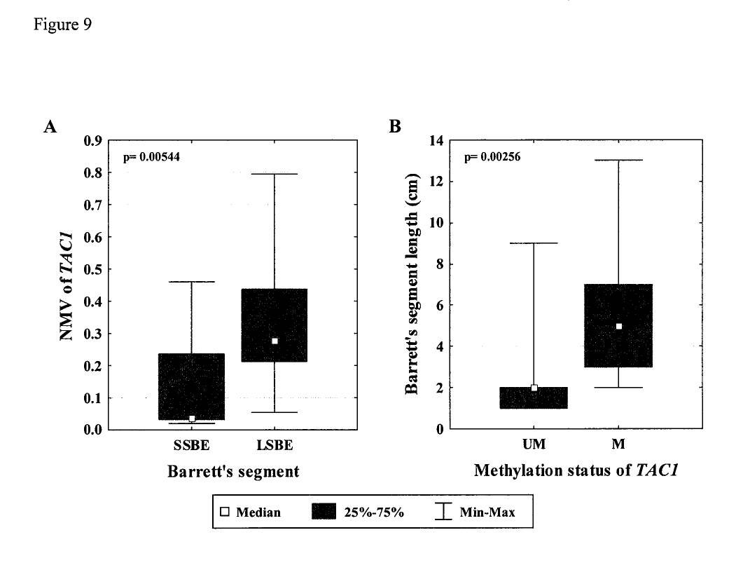

BE was defined as long-segment (LSBE) if it was equal to or greater than 3 cm in length, or short-segment (SSBE) if less than 3 cm, according to generally accepted criteria. The mean NMV of TAC1 was significantly higher in LSBE (mean=0.339) than in SSBE (mean=0.119; p=0.00256, Student's t-test, FIG. 9A). Similarly, the segment lengths of BEs with hypermethylated TAC1 promoters were significantly longer than the segment lengths of BEs with unmethylated promoters (mean=5.56 vs. 2.25 cm; p=0.00544, Student's t-test; FIG. 9B), and the frequency of TAC1 hypermethylation was significantly higher in LSBE than in SSBE (97.5% vs. 28.6%; p=0.0022, Fisher's exact test).

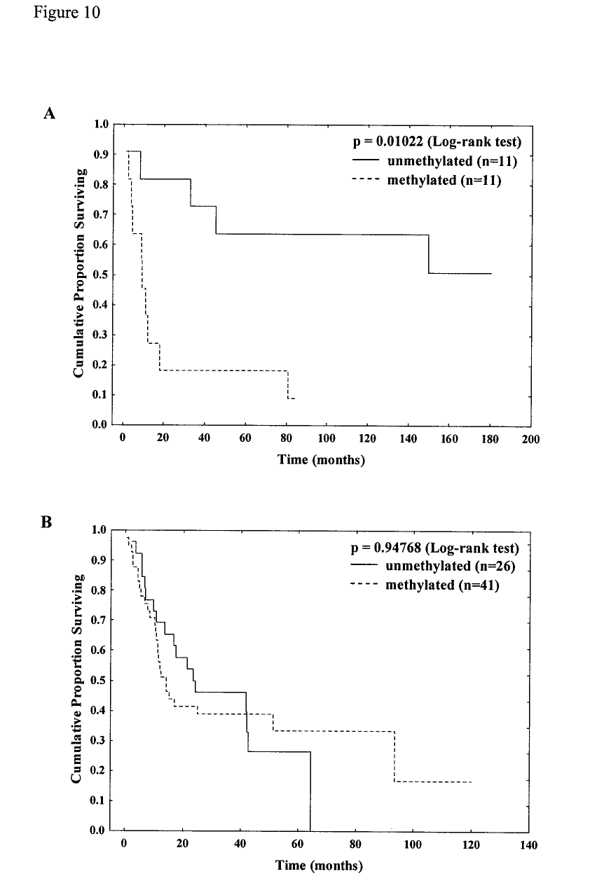

Overall patient survival also correlated with TAC1 methylation status in ESCC patients, but not in EAC patients (FIG. 10). ESCC patients with hypermethylation of TAC1 had significantly shorter survivals than did patients without TAC1 methylation (mean 22 months vs. 110 months; p=0.0102, log-rank test; FIG. 10A).

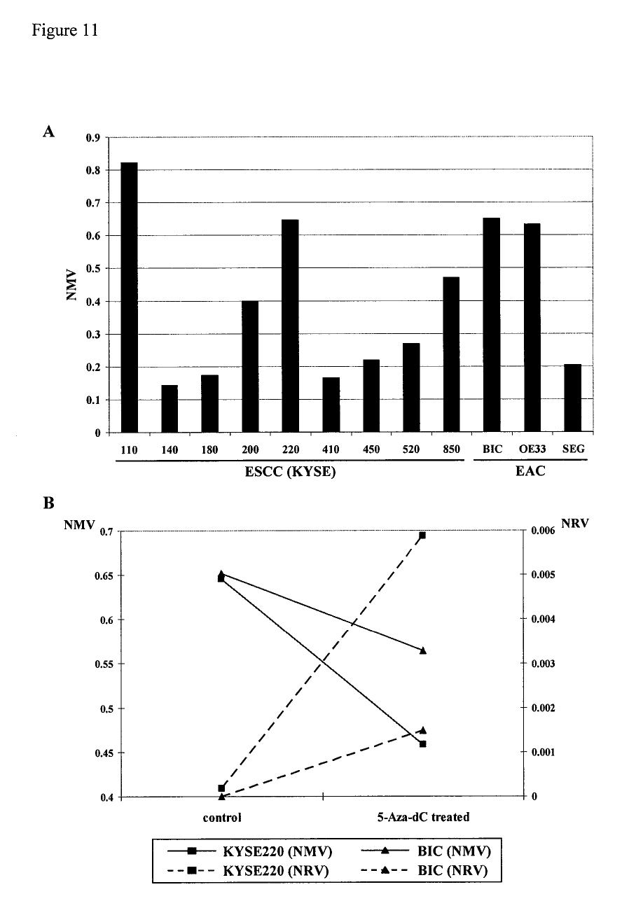

All 12 (3 EAC, 9 ESCC) esophageal cancer cell lines showed high TAC1 NMV levels above the cutoff level of 0.12 (FIG. 11A). KYSE 220 and BIC, which exhibited the highest NMVs among the ESCC and EAC cell lines, respectively, were subjected to 5-Aza-dC treatment. After 5-Aza-dC treatment, the NMV of TAC1 was diminished, while the mRNA level of TAC1 was increased, in both KYSE220 and BIC cells (FIG. 11B).

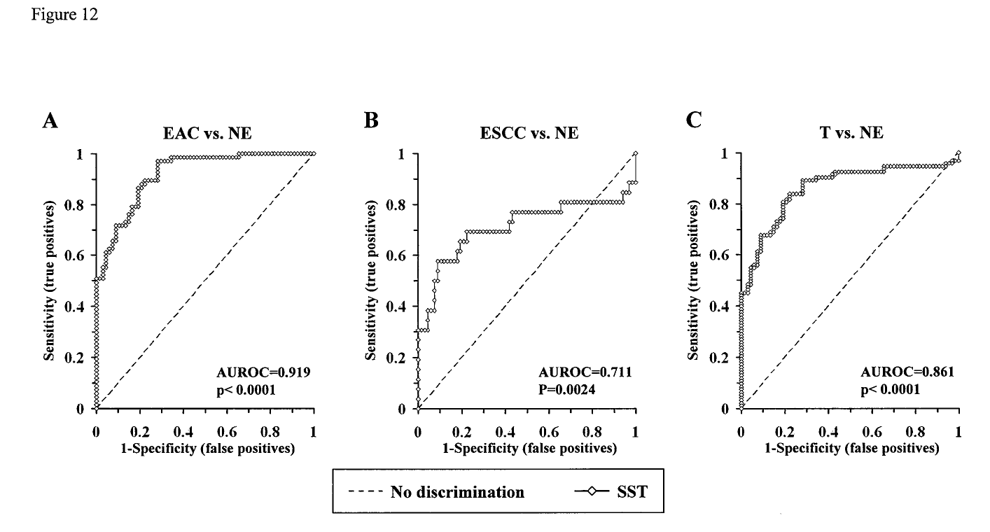

Promoter hypermethylation of the SST gene was analyzed in 67 NE, 60 BE (36 Ba and 24 Bt), 19 LGD, 21 HGD, 67 EAC and 26 ESCC. SST promoter hypermethylation showed highly discriminative ROC curve profiles, which clearly distinguished both ESCC (p=0.0024) and EAC (p<0.0001) from NE. ROC curves with corresponding AUROCs for SST of ESCC vs. NE, EAC vs. NE, and combined esophageal cancer (T) vs. NE are shown in FIG. 12.

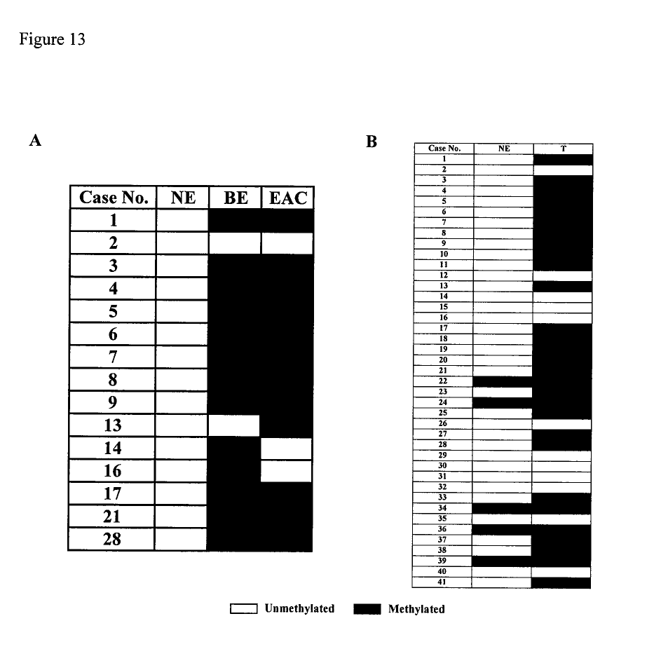

The cutoff NMV for SST (0.1) was chosen from the ROC curves to maximize sensitivity and specificity. Fourteen (53.8%) of 26 ESCCs exhibited hypermethylation of SST. The NMV of SST was significantly higher in ESCC, EAC, HGD, LGD, and BE than in NE (p<0.0000001, Student's t-test). Incremental increases in the frequency of SST hypermethylation were observed during progression from NE (9%) to BE (70%), HGD (71.4%), and EAC (71.6%), whereas LGD (63.2%) showed a slightly lower frequency of SST hypermethylation than did BE or HGD. Both SST hypermethylation frequency and mean NMV were higher in Bt than in Ba (83.3% vs. 61.1% and 0.2763 vs. 0.2431, respectively), with the difference in frequency just barely falling short of statistical significance (p=0.058, Fisher's exact test). Among 15 cases with corresponding NE, BE and EAC, one (No. 2) was unmethylated, one (No. 13) was methylated only in EAC, two (Nos. 14 and 16) were methylated only in BE, and the remaining eleven were methylated in both BE and EAC (FIG. 13A). Among 41 cases with corresponding NE and T, five (100%) of five cases (Nos. 22, 24, 34, 36 and 39) showing methylation in NE were also methylated in corresponding EAC (FIG. 13B), and the SST NMVs in T (mean=0.231) were significantly higher than those in corresponding NE (mean=0.055) (p<0.0000001, Student's paired t-test).

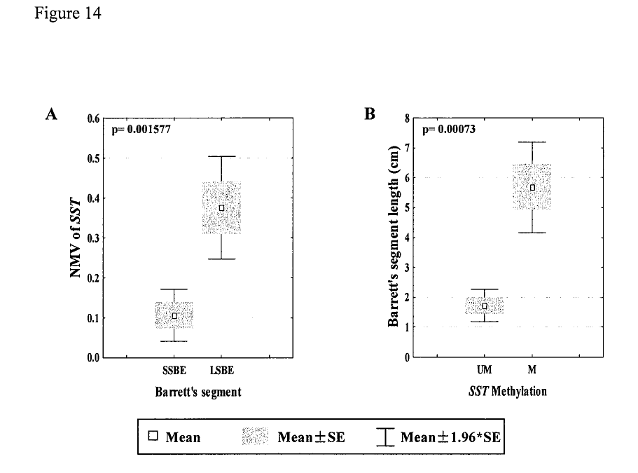

BE was defined as long-segment (LSBE) if it was equal to or greater than 3 cm in length, or short-segment (SSBE) if less than 3 cm, according to generally accepted criteria. The mean NMV of SST was significantly higher in LSBE (mean=0.375) than in SSBE (mean=0.106; p=0.00158, Student's t-test; FIG. 14A). Similarly, the segment lengths of BEs with methylated SST promoters (mean=5.68 cm) were significantly longer than the segment lengths of BEs with unmethylated SST promoters (mean=1.73 cm; p=0.00073, Student's t-test; FIG. 14B); and the frequency of SST hypermethylation was significantly higher in LSBE than in SSBE (p=0.0004, Fisher's exact test).

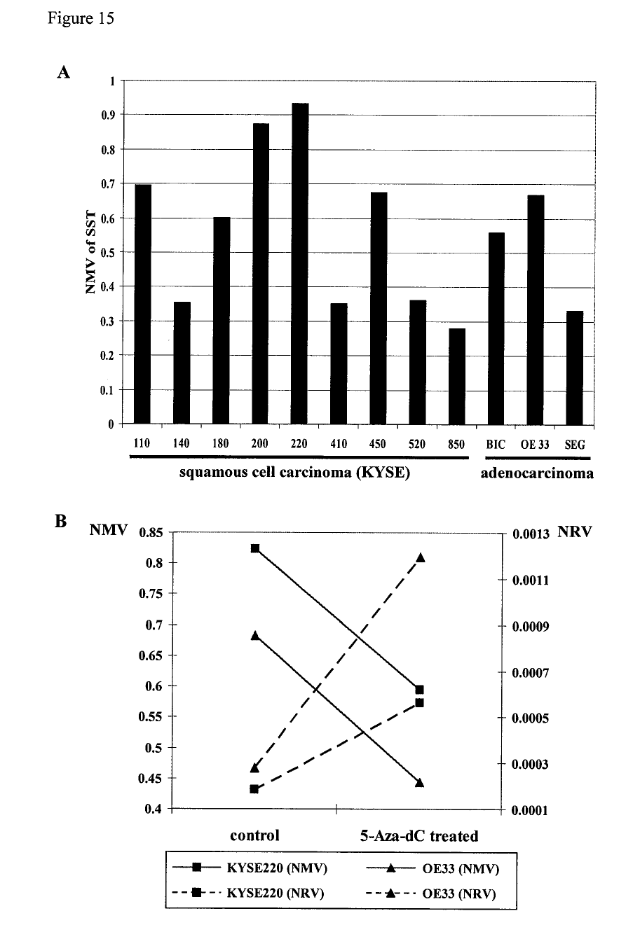

All 12 (3 EAC, 9 ESCC) esophageal cancer cell lines showed high SST NMV levels, greater than the cutoff level of 0.1 (FIG. 4A). KYSE 220 and OE33, which exhibited the highest NMVs among the ESCC and EAC cell lines, respectively, were subjected to 5-Aza-dC treatment. After 5-Aza-dC treatment, the NMV of SST was diminished, while the mRNA level of SST was increased, in both KYSE220 and OE33 cells (FIG. 15B).

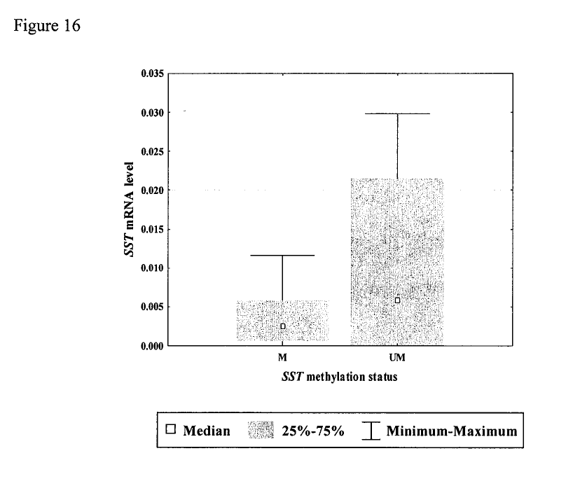

To elucidate the relationship between DNA hypermethylation and mRNA expression of SST, we further determined SST mRNA levels in 22 EAC samples using real-time RT-PCR. The SST mRNA levels of EACs with unmethylated SST promoters were significantly higher than those of EACs with methylated SST promoters (p=0.047, Student's t test; FIG. 16).

### EXAMPLE 3

Sample and Cell Line Selection

Of 259 specimens that were examined, 66 were normal esophageal specimens [N, including 19 obtained from non-Barrett's/non-esophageal cancer patients (NE), 20 were from ESCC patients (NEcS), and 27 were from EAC patients (NEcA)]. In addition, 60 specimens were from nondysplastic Barrett's metaplasias without dysplasia {BE, including 36 obtained from patients with Barrett's aloneonly (Ba) and 24 BE obtained from patients with Barrett's accompanied by EAC (Bt)}, 40 were dysplastic Barrett's specimens occurring in BE {D, including 19 low-grade (LGD) and 21 high-grade (HGD)}, 67 were EACs, and 26 were ESCCs. Research tissues were obtained from grossly apparent Barrett's epithelium or from mass lesions in patients manifesting these changes at endoscopic examination, and histology was confirmed using parallel aliquots taken obtained from identical locations at endoscopy. All biopsy specimens were stored in liquid nitrogen before DNA extraction. Three esophageal adenocarcinoma (BIC, OE33 and SEG) and nine esophageal squamous cell carcinoma (KYSE 110, 140, 180, 200, 220, 410, 450, 520 and 850) cell lines were also examined. All cells were cultured in 47.5% RPMI 1640, 47.5% F-12 supplemented with 5% fetal bovine serum.

Sample Treatment

Samples were treated as in Examples 1 and 2. Briefly, to determine AKAP12 mRNA levels, one-step real-time quantitative RT-PCR was performed using a Qiagen QuantiTect Probe RT-PCR Kit (Qiagen, Hilden, Germany) and the ABI 7700 Sequence Detection System (Applied Biosystems, Foster City, Calif.). Primers and probe for AKAP12 were as follows:

| | |
|---|---|
| AKAP12-forward: | 5′-CGCCACCAAGCTCCTACA-3′, |
| AKAP12-reverse: | 5′-GCCATTTAGGTCACCCTCCTG-3′ and |
| probe: | 5′-AAGAATGGTCAGCTGTCCACCATCA-3′. |

β-actin was used for normalization of data. Primers and probe for β-actin were the same as previously reported. A standard curve was generated using serial dilutions of qPCR Reference Total RNA (Clontech, Mountainview, Calif.). Normalized mRNA value (NRV) was calculated according to the following formula for relative expression of target mRNA: NRV=(TarS/TarC)/(ACTB-S/A CTB-C, where TarS and TarC represent levels of mRNA expression for the target gene in sample and control mRNAs, respectively, while ACTB-S and ACTB-C correspond to amplified ACTB levels in sample and control mRNAs, respectively.

Statistical Analysis

Receiver-operator characteristic (ROC) curve analysis was performed using NMVs for the 67 EAC, 26 ESCC and 66 N specimens by Analyse-it© software (Version 1.71, Analyse-it Software, Leeds, UK). Using this approach, the area under the ROC curve (AUROC) identified optimal sensitivity and specificity levels at which to distinguish normal from malignant esophageal tissues, yielding corresponding NMV thresholds defining methylation status of AKAP12. The threshold NMV value determined from this ROC curve was applied to determine the status of AKAP12 methylation in all tissue types included in the present study. For all other statistical tests, Statistica (version 6.1; StatSoft, Inc., Tulsa, Okla.) was used. Differences with p<0.05 were considered significant.

Diagnostic Markers for Barrett's Associated Esophogeal Neoplastic Progression

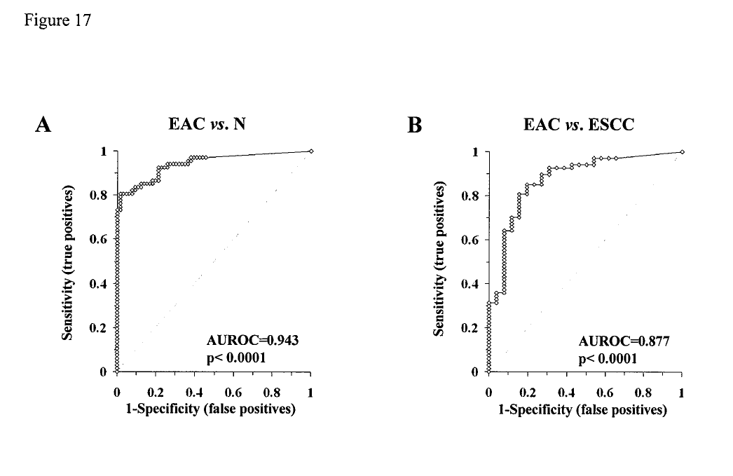

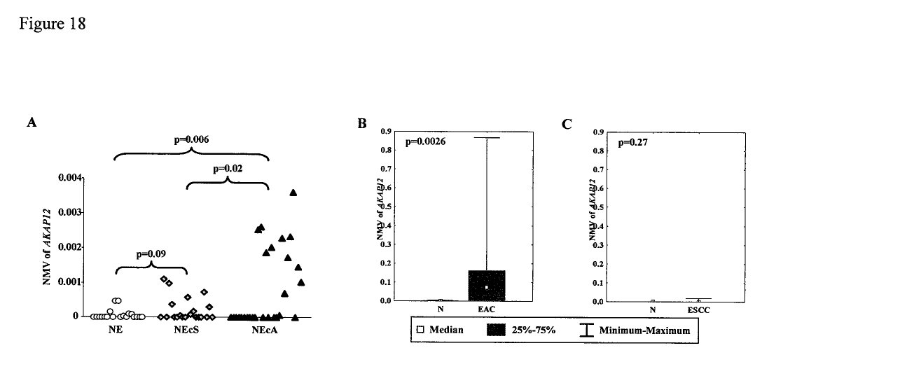

AKAP12 promoter hypermethylation showed highly discriminative ROC curve profiles, clearly distinguishing EAC from both N and ESCC (p<0.0001; FIG. 17). The cutoff NMV for AKAP12 (0.05) was chosen based upon the ROC curve analysis to discriminate T (i.e., EAC and ASCC) from N to maximize both sensitivity and specificity. NMVs of AKAP12 were significantly higher in ESCC, EAC, D, HGD, LGD, BE, Ba and Bt than in N (p<0.05, Student's t-test). Moreovere, the mean NMVs of AKAP12 was significantly higher in NEcA (mean=0.00082) than in N (mean=0.00007 and p=0.006) or NEcS (mean=0.00021 and p=0.02), but not significantly higher in NEcS vs. N (p=0.09; Student's t-test; FIG. 18A). The frequency of AKAP12 hypermethylation was increased in Ba (38.9%), D (52.5%), and EAC (52.2%) vs. N (0%; p<0.05, Student's t-test). Both AKAP12 hypermethylation frequency and mean NMV were higher in Bt than in Ba (62.5% vs. 38.9% and 0.1216 vs. 0.1122, respectively). However, these differences were not statistically significant (P>0.05, Student's t-test). The mean AKAP12 NMVs of EAC (0.1241) was significantly higher than those of corresponding N (0.0008) in 27 cases with corresponding N and EAC (p=0.0026, Student's paired t-test; FIG. 18B). In contrast to EAC, only two (7.7%) of 26 ESCCs showed hypermethylation of AKAP12. There was no significant difference between tumor and normal tissue mean AKAP12 NMVs in 13 cases with matching ESCC (0.0018) and N (0.0002; p=0.27, Student's paired t-test; FIG. 18C). Both AKAP12 hypermethylation frequency and mean NMV were significantly higher in EAC than in ESCC (52.2% vs. 7.7%, p<0.0001 and 0.1157 vs. 0.01, p=0.0013, respectively), as well as in D vs. in ESCC (52.5% vs. 7.7%, p=0.0002 and 0.1344 vs. 0.01, p=0.0013, respectively), and in BE vs. in ESCC (48.3% vs. 7.7%, p=0.0002 and 0.116 vs. 0.01, p=0.0008;).

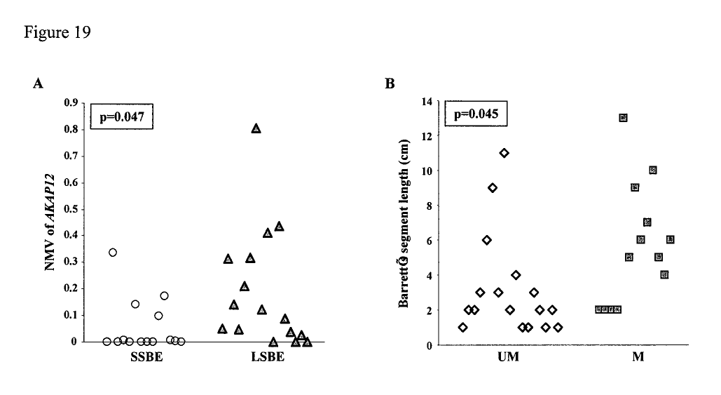

According to generally accepted criteria, BE was defined as long-segment (LSBE) if it was equal to or greater than 3 cm in length, or short-segment (SSBE) if less than 3 cm. The mean NMV of AKAP12 was significantly higher in LSBE (0.1879) than in SSBE (0.0543; p=0.047, Student's t-test, FIG. 19A). Similarly, segment lengths of BEs with methylated AKAP12 promoters (mean=5.62 cm) were significantly longer than segment lengths of BEs with unmethylated AKAP12 promoters (3.18 cm; p=0.045, Student's t-test; FIG. 19B), and the frequency of AKAP12 hypermethylation was higher in LSBE than in SSBE (56.3% vs. 28.6%, p=0.16, Fisher's exact test).

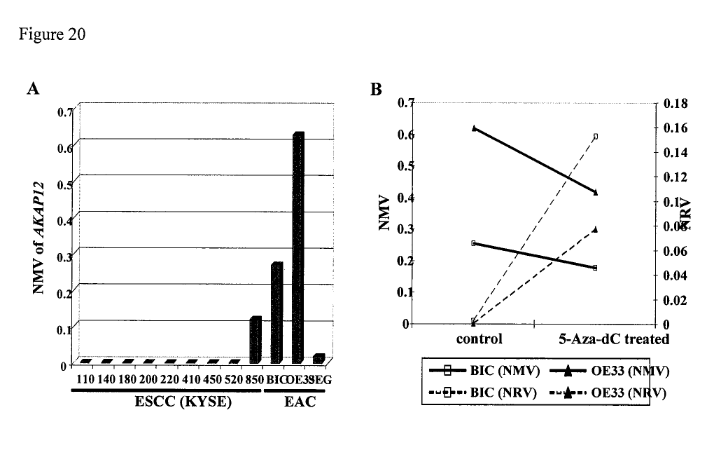

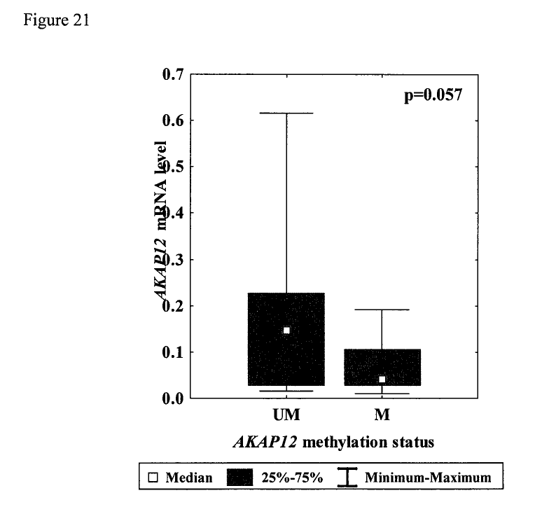

One of nine ESCC and two of three EAC esophageal cancer cell lines showed high AKAP12 methylation levels, above the threshold level of 0.05 (FIG. 20A). BIC and OE33 cells were subjected to 5-Aza-dC treatment. After 5-Aza-dC treatment, the NMV of AKAP12 was diminished and the mRNA level of AKAP12 was increased in both cell lines (FIG. 20B). In addition, the AKAP12 mRNA levels of EACs with unmethylated AKAP12 promoters (mean=0.1663) were higher than those of EACs with methylated AKAP12 (mean=0.0668), with this difference barely failing to achieve statistical significance (p=0.057, Student's t test; FIG. 21.

What is claimed is:

1. A method for diagnosing the presence of an abnormal condition in a subject suspected of having an abnormal condition, said method comprising
a) determining a methylation status of at least one gene in the subject; and
b) comparing the methylation status of said at least one gene in said subject to the normal methylation status of said at least one gene;
wherein a difference between the methylation status of said at least one gene in said subject and the normal methylation status of said at least one gene is indicative of the presence or absence or colon cancer in the subject, and wherein said at least one gene is tachykinin-1 (TAC1).

2. The method of claim 1, wherein the abnormal condition is colon cancer, esophageal cancer or gastric cancer.

3. The method of claim 2, further comprising determining the methylation status of the somatostatin gene (SST).

4. The method of claim 3, wherein said determining said methylation status comprises using an assay selected from the group consisting of Southern blotting, single nucleotide primer extension, methylation-specific polymerase chain reaction (MSP), restriction landmark genomic scanning for methylation (RLGS-M), CpG island microarray, SNUPE, and COBRA.

5. The method of claim 3, wherein the methylation status of a panel of genes is determined and compared to the normal methylation status of said panel of genes.

6. The method of claim 5, wherein said panel comprises three or more genes.

7. The method of claim 6, wherein said panel comprises at least 4 or 5 genes.

8. The method of claim 7, wherein said panel comprises at least 5 genes.

9. The method of claim 8, wherein said panel comprises TAC1, SST, nel-like type 1 (NELL1), A kinase [PRKA] anchor protein [gravin] 12 (AKAP12), caveolin, endoglin and T-lymphocyte maturation associated protein (MAL).

10. A method of monitoring the development of an abnormal condition in a subject, said method comprising
a) determining a methylation status of at least one gene in said subject at a first and second time point; and
b) determining a difference between said methylation status at said first and second time points to assess a change of methylation status over time;
wherein said difference over time is indicative of a change in the subject's probability of developing said abnormal condition, and wherein said at least one gene comprises tachykinin-1 (TAC1).

11. The method of claim 10, wherein the abnormal condition is colon cancer, esophageal cancer or gastric cancer.

12. The method of claim 11, further comprising determining the methylation status of the somatostatin gene (SST).

13. The method of claim 12, wherein said determining said methylation status comprises using an assay selected from the group consisting of Southern blotting, single nucleotide primer extension, methylation-specific polymerase chain reaction (MSP), restriction landmark genomic scanning for methylation (RLGS-M), CpG island microarray, SNUPE, and COBRA.

14. The method of claim 12, wherein the methylation status of a panel of genes is determined and compared to the normal methylation status of said panel of genes.

15. The method of claim 14, wherein said panel comprises three or more genes.

16. The method of claim 15, wherein said panel comprises at least 4 or 5 genes.

17. The method of claim 16, wherein said panel comprises at least 5 genes.

18. The method of claim 17, wherein said panel comprises TAC1, SST, nel-like type 1 (NELL1), A kinase [PRKA] anchor protein [gravin] 12 (AKAP12), caveolin, endoglin and T-lymphocyte maturation associated protein (MAL).

19. A method for assessing the probability of a subject having an abnormal condition, said method comprising
a) determining a methylation status of at least one gene in gross normal tissue of the subject; and
b) comparing the methylation status of said at least one gene in said subject to the normal methylation status of said at least one gene;
wherein a difference between the methylation status of said at least one gene in said gross normal tissue of said subject and the normal methylation status of said at least one gene indicates that the subject has an altered probability of having said abnormal condition, wherein said at least one gene is tachykinin-1 (TAC1).
